SEQUENCE LISTING (33 sequences)

SEQ ID NO: 1 - protein, 101 aa, synthetic construct
  VARIANT 23..31: note = Any amino acid
  VARIANT 52..56: note = Any amino acid
  VARIANT 76..86: note = Any amino acid
  REGION 1..101: note = General Fn3 scaffold
  REGION 23..31: note = BC loop
  REGION 52..56: note = DE loop
  REGION 76..86: note = FG loop
SSDSPRNLEVTNATPNSLTISWXXXXXXXXXYRITYGETGGNSPSQEFTVPXXXXXATISGLKPGQDYTITVYAVXXXXXXXXXXXPISINYRTEIDKPSQ

SEQ ID NO: 2 - protein, 97 aa, synthetic construct
  REGION 1..97: note = anti-mesothelin Fn3 1.4.1
  REGION 23..31: note = BC loop
  REGION 52..56: note = DE loop
  REGION 76..83: note = FG loop
SSDSPRNLEVTNATPNSLTISWDYPRYNSLYYRITYGETGGNSPSQEFTVPRYYSYATISGLNPGQDYTITVYAVSGGSTSNPISINYRTEIDKPSQ

SEQ ID NO: 3 - protein, 98 aa, synthetic construct
  REGION 1..98: note = anti-mesothelin Fn3 2.4.1
  REGION 23..31: note = BC loop
  REGION 52..56: note = DE loop
  REGION 76..83: note = FG loop
SSDSPRNLEVTNATPNSLTISWDYPRYNSLYYRITYGETGGNSPSQEFTVPRYYSYATISGLNPGQDYTITVYAVSRYNHYSNPISINYRTEIDKPSQ

SEQ ID NO: 4 - protein, 98 aa, synthetic construct
  REGION 1..98: note = anti-mesothelin Fn3 3.4.4
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYSYATISGLNPGQDYTITVYAVSRYNHYSNPISINYRTEIDKPSQ

SEQ ID NO: 5 - protein, 98 aa, synthetic construct
  REGION 1..98: note = anti-mesothelin Fn3 3.4.5
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYSYATISGLNSGQDYTITVYAVSRYNHYLNPISINYRTEINKPSQ

SEQ ID NO: 6 - protein, 98 aa, synthetic construct
  REGION 1..98: note = anti-mesothelin Fn3 4.4.8
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYLYATVSGLSPGQDYTITVYAVSRYNHYSNPISINYRTEIDEPSQ

SEQ ID NO: 7 - protein, 98 aa, synthetic construct
  REGION 1..98: note = anti-mesothelin Fn3 5.3.1
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYPNPISINYRTEIDKPSQ

SEQ ID NO: 8 - protein, 98 aa, synthetic construct
  REGION 1..98: note = anti-mesothelin Fn3 5.3.2
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSNPISINYRTEIDKPSQ

SEQ ID NO: 9 - protein, 98 aa, synthetic construct
  REGION 1..98: note = anti-mesothelin Fn3 5.3.19
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYLYATISGLKPGQDYTITVYAVSRYNHYSNPISINYRTEIDKPSQ

SEQ ID NO: 10 - protein, 98 aa, synthetic construct
  REGION 1..98: note = anti-mesothelin Fn3 5.3.8
  REGION 23..31: note = BC loop
  REGION 52..56: note = DE loop
  REGION 76..83: note = FG loop
SSDSPRNLEVTNATPNFLTISWDYPRYNALYYRITYGETGGNSPSQEFTVPRYYLYATISGLKPGQDYTITVYAVSRYNHYSNPISINYRTEIDKPSQ

SEQ ID NO: 11 - protein, 98 aa, synthetic construct
  REGION 1..98: note = anti-mesothelin Fn3 5.3.9
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYLYATISGLKPGQDYTITVYAVSRYNHYPNPISINYRTEIDKPSQ

SEQ ID NO: 12 - protein, 98 aa, synthetic construct
  REGION 1..98: note = anti-mesothelin Fn3 5.3.11
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYSHATVSGLNPGQDYTITVYVVSRYNHYPNPVSINYRTEIGKPSQ

SEQ ID NO: 13 - protein, 98 aa, synthetic construct
  REGION 1..98: note = anti-mesothelin Fn3 5.3.12
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYPSPISINYRTEIDKPSQ

SEQ ID NO: 14 - protein, 98 aa, synthetic construct
  REGION 1..98: note = anti-mesothelin Fn3 5.3.17
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSSPISINYRTEIDKPSQ

SEQ ID NO: 15 - protein, 98 aa, synthetic construct
  REGION 1..98: note = anti-mesothelin Fn3 5.3.18
  REGION 23..31: note = BC loop
  REGION 52..56: note = DE loop
  REGION 76..83: note = FG loop
SSYSPRNLEVTDATPNSLTISWDYPRYNALYYRITYGETGGNSPSQEFTVPRYYLYATISGLKPGQDYTITVYAVSRYNHYSSPISINYRTEIDKPSQ

SEQ ID NO: 16 - protein, 98 aa, synthetic construct
  REGION 1..98: note = anti-mesothelin Fn3 5.3.3
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHHSNPISINYRTEIDKPSQ

SEQ ID NO: 17 - protein, 98 aa, synthetic construct
  REGION 1..98: note = anti-mesothelin Fn3 5.3.5
  REGION 23..31: note = BC loop
  REGION 52..56: note = DE loop
  REGION 76..83: note = FG loop
SSDSPRNLEVTNATPNSLTISWDYPRYNALHYRITYGETGGNSPSQEFTVPRYYLYATISGLKPGQDYTITVYAVSRYSHYSNPISINYRTEIDKPSQ

SEQ ID NO: 18 - protein, 98 aa, synthetic construct
  REGION 1..98: note = anti-mesothelin Fn3 5.3.7
  REGION 23..31: note = BC loop
  REGION 52..56: note = DE loop
  REGION 76..83: note = FG loop
SSDSPRNLEVTNATPNSLTISWDYPRYNARYYRITYGETGGNSPSQEFTVPRYYLYATISGLKPGQDYTITVYAVSRYTNYSNPISINYRTEIDKPSQ

SEQ ID NO: 19 - protein, 98 aa, synthetic construct
  REGION 1..98: note = anti-mesothelin Fn3 5.3.15
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNGVSNPISINYRTEIDKPSQ

SEQ ID NO: 20 - protein, 9 aa, synthetic construct
  REGION 1..9: note = BC loop
DYPRYNALY

SEQ ID NO: 21 - protein, 9 aa, synthetic construct
  REGION 1..9: note = BC loop
DYPRYNALH

SEQ ID NO: 22 - protein, 9 aa, synthetic construct
  REGION 1..9: note = BC loop
DYPRYNARY

SEQ ID NO: 23 - protein, 5 aa, synthetic construct
  REGION 1..5: note = DE loop
RYYLY

SEQ ID NO: 24 - protein, 5 aa, synthetic construct
  REGION 1..5: note = DE loop
RYYSH

SEQ ID NO: 25 - protein, 8 aa, synthetic construct
  REGION 1..8: note = FG loop
SRYNHYSN

SEQ ID NO: 26 - protein, 8 aa, synthetic construct
  REGION 1..8: note = FG loop
SRYNHYPN

SEQ ID NO: 27 - protein, 8 aa, synthetic construct
  REGION 1..8: note = FG loop
SRYNHYPS

SEQ ID NO: 28 - protein, 8 aa, synthetic construct
  REGION 1..8: note = FG loop
SRYNHYSS

SEQ ID NO: 29 - protein, 8 aa, synthetic construct
  REGION 1..8: note = FG loop
SRYNHHSN

SEQ ID NO: 30 - protein, 8 aa, synthetic construct
  REGION 1..8: note = FG loop
SRYSHYSN

SEQ ID NO: 31 - protein, 8 aa, synthetic construct
  REGION 1..8: note = FG loop
SRYTNYSN

SEQ ID NO: 32 - protein, 8 aa, synthetic construct
  REGION 1..8: note = FG loop
SRYNGVSN

SEQ ID NO: 33 - protein, 6 aa, synthetic construct
HHHHHH

(19) United States
(12) Patent Application Publication
(10) Pub. No.: US 2026/0146076 A1
(43) Pub. Date: May 28, 2026

(54) Mesothelin-Binding Engineered Fibronectin Type III Protein Scaffold

(71) Applicant: Smith College, Northampton, MA (US)

(72) Inventors: Sarah J. Moore, Amherst, MA (US); Allison R. Sirois, Berlin, CT (US)

(21) Appl. No.: 19/397,094

(22) Filed: Nov. 21, 2025

Related U.S. Application Data
(60) Provisional application No. 63/723,747, filed on Nov. 22, 2024.
(60) Provisional application No. 63/872,574, filed on Aug. 29, 2025.

(51) Int. Cl. C07K 14/78; A61K 38/00

(52) U.S. Cl. CPC C07K 14/78; A61K 38/00

(57) ABSTRACT

Mesothelin-binding fibronectin type III (Fn3) domain proteins having improved mesothelin binding characteristics are described.

### CROSS-REFERENCE TO RELATED APPLICATIONS

The present application claims priority from U.S. Provisional Application No. 63/723,747, filed Nov. 22, 2024, and U.S. Provisional Application No. 63/872,574, filed Aug. 29, 2025. The contents of each of the foregoing applications are hereby incorporated by reference herein in their entirety for all purposes.

### GOVERNMENT RIGHTS IN INVENTION

This invention was made with government support under R15CA198927-01 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 20, 2025, is named 4634_1014_SL.xml and is 65,141 bytes in size.

### TECHNICAL FIELD

The present invention relates to mesothelin-binding fibronectin type III tenth domain (Fn3) proteins, and more particularly to Fn3 proteins having improved mesothelin binding characteristics.

### BACKGROUND ART

Mesothelin (MSLN) is a tumor biomarker expressed at high levels on the surface of numerous cancers, with extremely limited expression in healthy tissues. MSLN-targeting agents developed for diagnosis and therapy could have a significant impact on the management of MSLN-expressing cancers. Pleural mesothelioma (PM) is a deadly cancer that arises from mesothelial cells lining the pleura, predominantly linked to asbestos exposure. There are currently no effective treatments, and diagnosis occurs in late stages of disease due to the lack of clinical symptoms in early stages.

Due to its overexpression in various tumor types and the preferential location at the membrane site, MSLN represents an attractive molecule for targeted therapies.^{1,2} In addition, MSLN has a role in cancer cell adhesion and metastasis.^{3,4} A recent analysis of more than 12,000 tumors identified various cancer types that might be particularly well suited for anti-MSLN drugs such as ovarian carcinoma, mesothelioma, and adenocarcinomas of the pancreas, lung, stomach, esophagus, and the colorectum.⁵

Among these, pleural mesothelioma (PM) is an aggressive cancer arising from the mesothelial cells lining the pleura and it is predominantly associated with asbestos exposure.⁶ Despite its rarity, PM is a fatal cancer accounting for 26,278 new deaths out of 30,870 new cases in 2020.7 Diagnosis usually occurs 30-50 years after asbestos exposure and in late stages, because clinical signs are not evident in early disease.⁶ Additionally, PM is known to develop resistance to treatments, and surgery is available only for candidate PM cases.^{8,9} Therefore, PM therapy and early diagnosis are still a challenge, leading to poor prognosis with 8-14 months median survival for PM patients.¹⁰

In recent decades, measuring MSLN serum levels and applying MSLN-targeted therapies have been proposed to overcome PM challenges.^{11,12} Despite initial enthusiasm, success has been elusive using antibody-drug conjugates directed at MSLN for PM. A phase II clinical trial with antibody-drug conjugate anetumab ravtansine (BAY 94-9343) targeting MSLN in PM patients was not superior to treatment with chemotherapeutic vinorelbine, although the antibody-drug conjugate did have a clinically manageable safety profile.¹³ Similarly, clinical trials with amatuximab, originally referred to as MORAb-009, have yet to translate into any approved MSLN-targeted therapy.¹⁴⁻¹⁶ Efforts to improve amatuximab and its conjugates to achieve desired clinical outcomes are ongoing. 16-19 While multiple CAR T-cell therapies have been developed targeting MSLN, they have also encountered barriers to success.²⁰⁻²³ Shedding of MSLN from the cell surface has been proposed as one key barrier to overcome for therapies based on antibody and CAR T-cells,^{24,25} which require extended engagement of the therapy at the cancer cell surface to be effective. Recent reports describe MSLN-targeting antibodies and CAR T-cell formats that recognize a domain of MSLN adjacent to the cell membrane and that is not shed from the cell, which may be able to overcome some of the challenges for these classes of therapy.^{26,27}

### SUMMARY OF THE EMBODIMENTS

In accordance with one embodiment of the invention, a polypeptide comprising a modified human fibronectin type III (Fn3) domain, wherein the polypeptide specifically binds to human mesothelin, and wherein BC, DE, and FG loops of the modified Fn3 domain comprise, respectively, the amino acid sequences set forth in (a) SEQ ID NOs: 20, 23, and 25; (b) SEQ ID NOs: 20, 23, and 26; (c) SEQ ID NOs: 20, 24, and 26; (d) SEQ ID NOs: 20, 23, and 27; (e) SEQ ID NOs: 20, 23, and 28; (f) SEQ ID NOs: 20, 23, and 29; (g) SEQ ID NOs: 21, 23, and 30; (h) SEQ ID NOs: 22, 23, and 31; or (i) SEQ ID NOs: 20, 23, and 32. In some embodiments, non-loop regions the Fn3 domain comprise of amino acid sequences having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to corresponding non-loop regions of SEQ ID NO: 1. In another embodiment, non-loop regions the Fn3 domain comprise of amino acid sequences having 100% identity to corresponding non-loop regions of SEQ ID NO: 1.

In some embodiments, the polypeptide binds to mesothelin with a K_{D} of less than 100 nM. In another embodiment, the polypeptide binds to mesothelin with a K_{D} of less than 20 nM.

In accordance with another embodiment of the invention, a polypeptide, comprising a human fibronectin type III (Fn3) domain, that binds to mesothelin with a K_{D} of less than 20 nM, wherein the polypeptide comprises SEQ ID NO: 8.

In accordance with another embodiment of the invention, a polypeptide, comprising a human fibronectin type III (Fn3) domain, that binds to mesothelin with a K_{D} of less than 20 nM, wherein the polypeptide comprises SEQ ID NO: 7.

In accordance with yet another embodiment of the invention, a polypeptide, comprising a human fibronectin type III (Fn3) domain, that binds to mesothelin with a K_{D} of less than 20 nM, wherein the polypeptide consists of SEQ ID NO: 8.

In accordance with one embodiment of the invention, a polypeptide, comprising a human fibronectin type III (Fn3) domain, that binds to mesothelin with a K_{D} of less than 20 nM, wherein the polypeptide consists of SEQ ID NO: 7.

In accordance with another embodiment of the invention, a polypeptide comprising a human fibronectin type III (Fn3) domain, wherein (a) non-loop regions of the Fn3 domain comprise of amino acid sequences having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to corresponding non-loop regions of an Fn3 sequence selected from the group consisting of SEQ ID NOs: 6-19 and (b) BC, DE, and FG loops of the Fn3 domain comprise of amino acid sequences 100% identical to corresponding BC, DE, and FG loops of the Fn3 sequence. In some embodiments, the non-loop regions of the Fn3 domain comprise of amino acid sequences having 100% identity to the corresponding non-loop regions of the Fn3 sequence.

In accordance with one embodiment of the invention, a composition comprising the polypeptide and a pharmaceutically acceptable carrier.

In accordance with another embodiment of the invention, a kit comprising the polypeptide and instructions for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1A shows an Fn3 scaffold structure having three solvent exposed loops that form a binding interface for engineering interactions with target molecules (from PDB 1TTG). FIG. 1B is a Fn3 hydrophilic library general amino acid sequence (SEQ ID NO:1), wherein X indicates any amino acid, in accordance with an embodiment of the invention. BC, DE, and FG loops (shown, respectively, as contiguous sequences of any amino acid, i.e., “X”) vary in length. Shown are the lengths that were predominantly selected during rounds of sorting. FIG. 1C is a plot showing the results of equilibrium binding assays to measure apparent dissociation constants for yeast-surface displayed Fn3 variants and soluble Fc-MSLN, in accordance with an embodiment of the invention. Data for 1.4.1 (SEQ ID NO:2) and 2.4.1 (SEQ ID NO:3) modified from Sirois 2018. Data for 3.4.4 (SEQ ID NO:4) and 3.4.5 (SEQ ID NO:5) modified from Sirois 2020. FIG. 1D is an SDS-PAGE gel showing 5.3.2 (SEQ ID NO:8) was readily expressed and purified from bacterial culture, in accordance with embodiments of the invention. Fourth round variant 4.4.8 (SEQ ID NO:6) and fifth round variant 5.3.1 (SEQ ID NO:7) were also analyzed.

FIG. 2A is a flow cytometry scatter plot showing MSLN (Glu296-Gly580) expressed on the surface of yeast with mutations removing three N-linked glycans, in accordance with an embodiment of the invention. Expressed MSLN was detected by a c-myc tag C-terminal to MSLN. Surface displayed MSLN was recognized by an anti-MSLN antibody. FIG. 2B shows flow cytometry scatter plots demonstrating that displayed MSLN bound MUC16 and Fn3 5.3.2, but did not bind secondary anti-His6 antibody (“His6” disclosed as SEQ ID NO: 33) or non-binding control Fn3 RDG protein, in accordance with an embodiment of the invention.

FIG. 3 shows histograms of MSLN expression in the MSTO-211H wild-type cell line (left) and MSLN-overexpressing lines MSTO+MSLN clone 1 (center) and MSTO+MSLN clone 7 (right), in accordance with an embodiment of the invention. MSLN expression was evaluated through flow cytometry by incubating the cells with a rabbit anti-MSLN primary antibody and a goat anti-rabbit PE-conjugated secondary antibody. Unlabeled cells for each cell line were used as a reference.

FIG. 4A is a plot showing representative binding curves for Fn3 5.3.2 using the MSLN-negative MSTO-211H wild-type and the MSLN-overexpressing lines MSTO clone 1 and MSTO clone 7, in accordance with an embodiment of the invention. Each curve represents one out of the three experimental replicates. Data were fit to a sigmoidal curve, and K_{D} was calculated as the concentration of Fn3 5.3.2 yielding half of the maximum observed effect. MSTO clone 1: K_{D})=11±4 nM; MSTO clone 7: K_{D})=11±2 nM; MSTO WT: no binding detected. FIG. 4B is a dot plot showing results of flow cytometry carried out on MSTO clone 1 stained with a PE-conjugated anti-MSLN primary antibody, Fn3 5.3.2 (10 nM) and an AF488-conjugated anti-HisTag antibody, or the combination of these two treatments (Costaining). The x-axis reports green fluorescence intensity detected through a FITC filter; the y-axis reports red fluorescence intensity detected through a PE filter.

FIG. 5 shows confocal microscopy images demonstrating that Fn3 5.3.2 and MSLN co-localize and internalize into MSLN-positive cells, in accordance with an embodiment of the invention. MSLN-overexpressing MSTO clone 7 was evaluated using a Red-Texas filter (em.: 595 nm; ex.: 561 nm; red) to detect MSLN (stained with a PE-conjugated primary antibody, A, D, G) and FITC filter (em.: 525 nm; ex.: 488 nm) to detect Fn3 (stained with an AF488-conjugated anti-HisTag antibody; B, E, H). DAPI (blue) was used to stain the nucleus (C, F, I). Panel A-C represents cells incubated at 4° C. for 1 h, in which co-localization of the MSLN/Fn3 complex on the cell surface is visible. Panel D-F shows Fn3 internalization into punctate structures (red arrows) in PM cells, after 1 h incubation at 37° C. Panel G-I reports cells treated with an anti-MSLN antibody for 1 h incubation at 37° C., mostly localized on the cancer cells surface.

### DETAILED DESCRIPTION OF SPECIFIC EMBODIMENTS

Disclosed herein are engineered proteins based on the non-antibody fibronectin type III (Fn3) protein scaffold that bind MSLN with high affinity and specificity, using yeast surface display and directed evolution.

Definitions. As used in this description and the accompanying claims, the following terms shall have the meanings indicated, unless the context otherwise requires:

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The term “K_{D}),” as used herein in the context of a mesothelin-binding Fn3 protein binding to mesothelin means the dissociation equilibrium constant of a particular mesothelin-binding Fn3 protein interaction or the affinity of a mesothelin-binding Fn3 protein for a protein (e.g., mesothelin).

“Polypeptide” as used herein refers to any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D). Peptides of the present disclosure are proteins derived from the tenth type III domain of fibronectin that have been modified to bind specifically to human mesothelin.

Percent (%) amino acid sequence identity herein means the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR™) software. Those skilled in the art can readily determine appropriate parameters for measuring alignment, including any algorithms needed to achieve maximal alignment over the full-length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Fn3” and “human fibronectin type III” refers to a human fibronectin type III tenth domain protein. An Fn3 domain is small, monomeric, soluble, and stable. It lacks disulfide bonds and, therefore, is stable under reducing conditions. The overall structure of Fn3 resembles the immunoglobulin fold. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel 3-strands are arranged as two beta sheets that form a stable core, while creating two “faces” composed of the loops that connect the beta or beta-like strands. Loops AB, CD, and EF are located at one face (“the south pole”) and loops BC, DE, and FG are located on the opposing face (“the north pole”). There are at least 15 different Fn3 modules in human fibronectin, and while the sequence homology between the modules is low, they all share a high similarity in tertiary structure. U.S. Pat. No. 8,933,199 is hereby incorporated by reference for its disclosure of fibronectin type III tenth domain proteins.

As used herein, non-loop regions of a Fn3 domain means the regions of a Fn3 domain other than the BC loop, the DE loop, and the FG loop. Each non-loop region has a discrete and contiguous amino acid sequence. In comparing Fn3 domain sequences, a corresponding non-loop region of a first Fn3 domain means the homologous non-loop region of a second Fn3 domain, e.g., the first non-loop region of the first Fn3 domain corresponds with the first non-loop region of the second Fn3 domain.

“Specifically binds,” “specific binding,” “selective binding, and “selectively binds,” as used interchangeably herein in the context of mesothelin-binding Fn3 proteins refers to mesothelin-binding Fn3 proteins that exhibit affinity for human mesothelin, but do not significantly bind (e.g., less than about 10% binding) to a different polypeptide as measured by a technique available in the art such as, but not limited to, an equilibrium binding assay and/or a competitive binding assay (e.g., competition ELISA, BIACORE assay).

“Mesothelin” refers to human mesothelin.

The term “pharmaceutically acceptable carrier” means solvents, carrier agents, diluting agents and the like which are used in the administration of pharmaceutical compounds.

In some embodiments, a polypeptide of the present disclosure comprises a fibronectin type III (Fn3) domain that binds to mesothelin, wherein the BC, DE, and FG loops of the Fn3 domain comprise, respectively:
SEQ ID NOs: 20, 23, and 25;
SEQ ID NOs: 20, 23, and 26;
SEQ ID NOs: 20, 24, and 26;
SEQ ID NOs: 20, 23, and 27;
SEQ ID NOs: 20, 23, and 28;
SEQ ID NOs: 20, 23, and 29;
SEQ ID NOs: 21, 23, and 30;
SEQ ID NOs: 22, 23, and 31; or
SEQ ID NOs: 20, 23, and 32.

In some embodiments, each non-loop region of the Fn3 domain comprises an amino acid sequence having at least 80, 85, 86, 87, 88, 89, 90, 91, 92, 93, 94, 95, 96, 97, 98, 99, or 100% identity to a corresponding non-loop region of SEQ ID NO: 1.

In some embodiments, each non-loop region of the Fn3 domain consists of an amino acid sequence having at least 80, 85, 86, 87, 88, 89, 90, 91, 92, 93, 94, 95, 96, 97, 98, 99, or 100% identity to a corresponding non-loop region of SEQ ID NO: 1.

In some embodiments, the BC loop, and/or the DE loop, and/or the FG loop comprises one amino acid substitution, such as a conservative amino acid substitution. In another embodiment, the BC loop, and/or the DE loop, and/or the FG loop comprises one amino acid deletion. In some embodiments, the BC loop, and/or the DE loop, and/or the FG loop comprises one amino acid insertion.

In some embodiments, the present disclosure contemplates a polypeptide comprising a human fibronectin type III (Fn3) domain, wherein (a) each non-loop region of the Fn3 domain comprises an amino acid sequence having at least 80, 85, 86, 87, 88, 89, 90, 91, 92, 93, 94, 95, 96, 97, 98, 99, or 100% identity to a corresponding non-loop region of an Fn3 sequence selected from the group consisting of SEQ ID NOs: 6-19 and (b) BC, DE, and FG loops of the Fn3 domain comprise of amino acid sequences 100% identical to corresponding BC, DE, and FG loops of the Fn3 sequence.

In some embodiments, the present disclosure contemplates a polypeptide comprising a human fibronectin type III (Fn3) domain, wherein (a) each non-loop region of the Fn3 domain consists of an amino acid sequence having at least 80, 85, 86, 87, 88, 89, 90, 91, 92, 93, 94, 95, 96, 97, 98, 99, or 100% identity to a corresponding non-loop region of an Fn3 sequence selected from the group consisting of SEQ ID NOs: 6-19 and (b) BC, DE, and FG loops of the Fn3 domain consist of amino acid sequences 100% identical to corresponding BC, DE, and FG loops of the Fn3 sequence.

In some embodiments, the BC loop, and/or the DE loop, and/or the FG loop comprises one amino acid substitution, such as a conservative amino acid substitution. In another embodiment, the BC loop, and/or the DE loop, and/or the FG loop comprises one amino acid deletion. In some embodiments, the BC loop, and/or the DE loop, and/or the FG loop comprises one amino acid insertion.

In some embodiments, a mesothelin-binding polypeptide of the present disclosure binds to mesothelin with a K_{D} of less than 10 nM, 20 nM, 30 nM, 40 nM, 50 nM, 60 nM, 70 nM, 80 nM, 90 nM, or 100 nM. In some embodiments, the mesothelin-binding polypeptide comprises the amino acid sequence of SEQ ID NO: 7. In another embodiment, the mesothelin-binding Fn3 polypeptide consists of the amino acid sequence of SEQ ID NO: 7. In some embodiments, the mesothelin-binding Fn3 polypeptide comprises the amino acid sequence of SEQ ID NO: 8. In another embodiment, the mesothelin-binding Fn3 polypeptide consists of the amino acid sequence of SEQ ID NO: 8.

In another embodiment, the Fn3 domain comprises an amino acid sequence selected from the group consisting of SEQ ID NOs: 6-19.

In some embodiments, the polypeptide comprises an amino acid sequence selected from the group consisting of SEQ ID NOs: 6-19.

In another embodiment, the Fn3 domain consists of an amino acid sequence selected from the group consisting of SEQ ID NOs: 6-19.

In some embodiments, the polypeptide consists of an amino acid sequence selected from the group consisting of SEQ ID NOs: 6-19.

In some embodiments, a composition comprising a mesothelin-binding Fn3 polypeptide of the present disclosure and a pharmaceutically acceptable carrier is contemplated.

In another embodiment, a kit comprising a mesothelin-binding Fn3 polypeptide of the present disclosure, along with instructions for use, is contemplated.

### Example 1: Fn3 Protein Variant 5.3.2 Binds MSLN with High Affinity

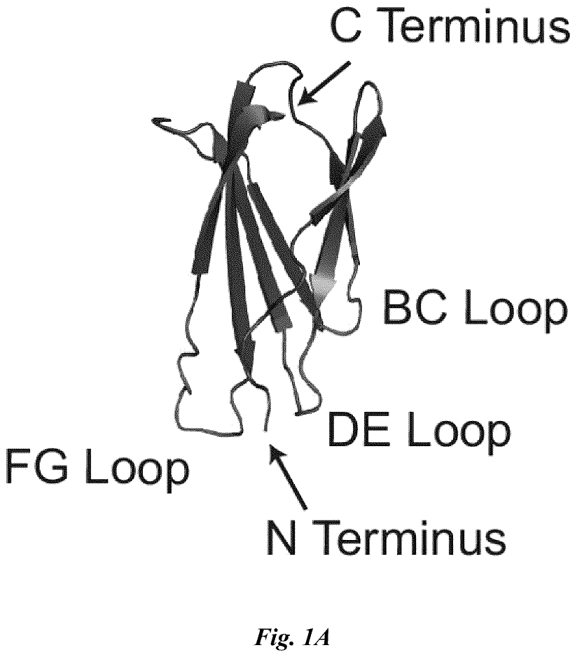

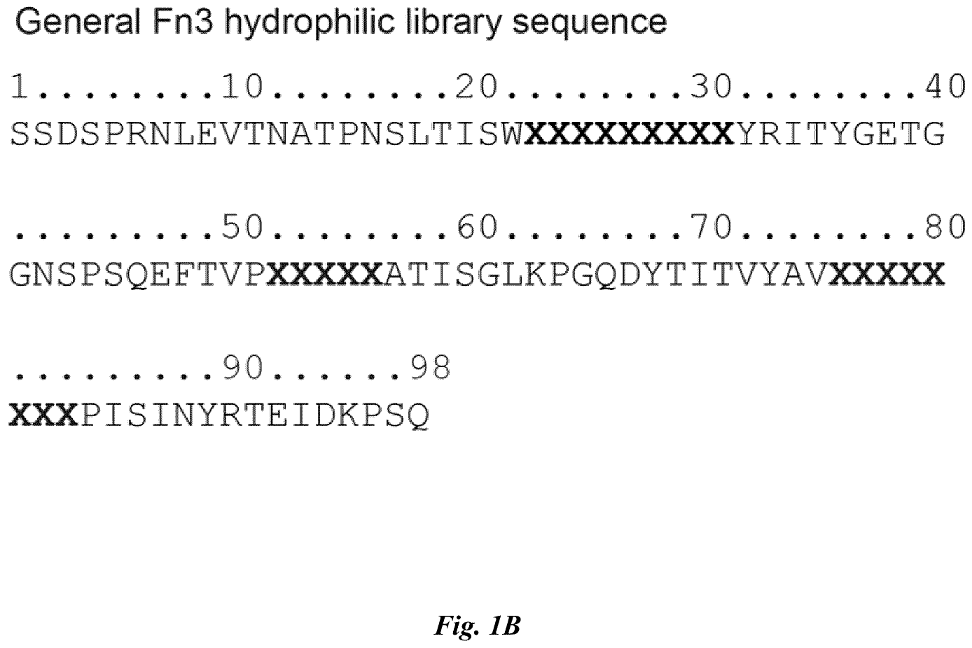

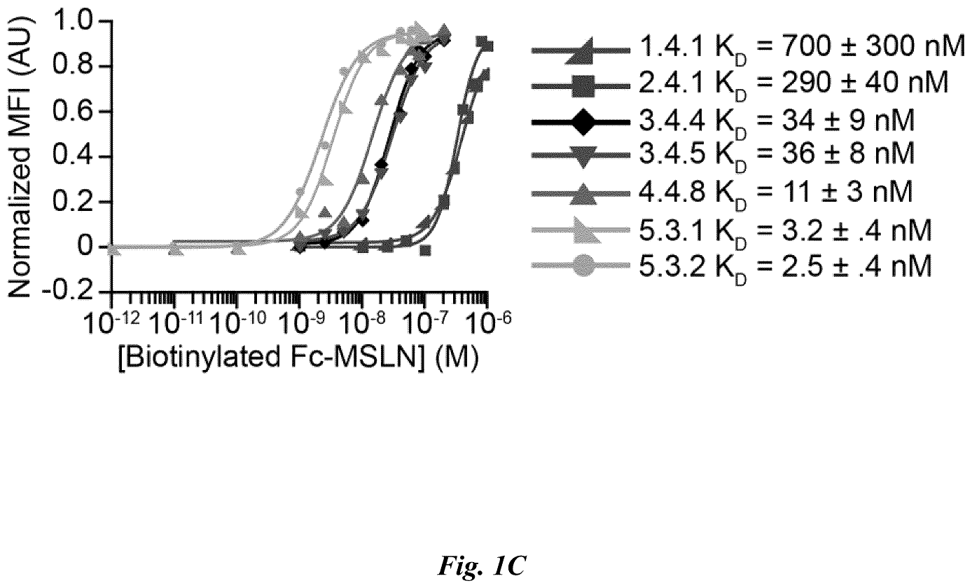

Previously, we reported engineering Fn3 variants that bind MSLN with dissociation constants (K_{D})) with moderate nanomolar (nM) affinities, and demonstrated that these variants were internalized and had a cytotoxic effect on MSLN-positive tumor cell lines. 58.59 Toward the goal of even stronger binding affinities that may be beneficial for clinical translation with in vivo applications, we continued with affinity maturation of our third generation Fn3 library (FIG. 1). The previously reported MSLN-binding Fn3 library was further mutated and affinity matured as fourth and fifth generation libraries using yeast surface display. Following mutagenesis, the fifth generation library was subjected to two rounds of MACS, a FACS sort for full-length expression, and three iterative rounds of dual-color FACS for binding to MSLN. The resultant population yielded enrichment of evident MSLN-binding clones and sequence analysis of individual clones identified two dominant variants, 5.3.1 (20% of sequenced variants) (SEQ ID NO:7) and 5.3.2 (25% of sequenced variants) (SEQ ID NO: 8) (FIG. 1C), in addition to 11 other sequences with low representation in the sequenced library (SEQ ID Nos: 9-19). All unique variants were tested for binding to Fc-MSLN using YSD with a single concentration of Fc-MSLN, and 5.3.1 and 5.3.2 exhibited greater binding signals compared to all other variants tested (data not shown). Of isolated sequences, variant 5.3.2 demonstrated the highest affinity to an Fc-MSLN based on equilibrium binding assays of enriched Fn3 variants analyzed from all completed rounds of affinity maturation (FIG. 1C) and exhibited high recombinant expression and ease of purification (FIG. 1D). Therefore, we pursued Fn3 5.3.2 for additional characterization and bioconjugate modification towards nuclear medicine applications, particularly for PM.

### Example 2: Fn3 5.3.2 Binds MSLN without Requiring N-Linked Glycosylation

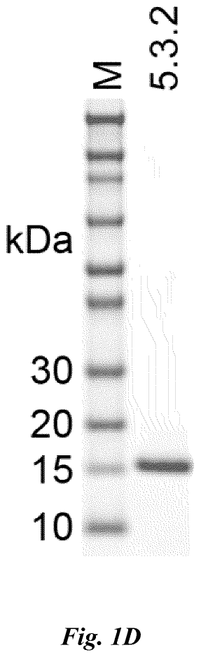

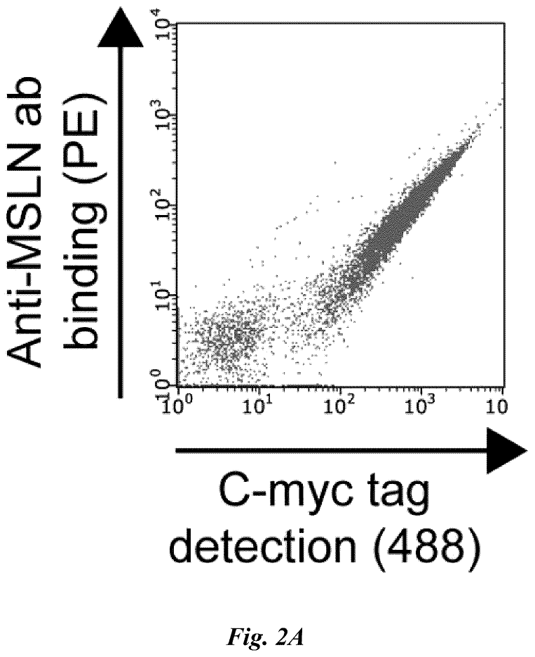

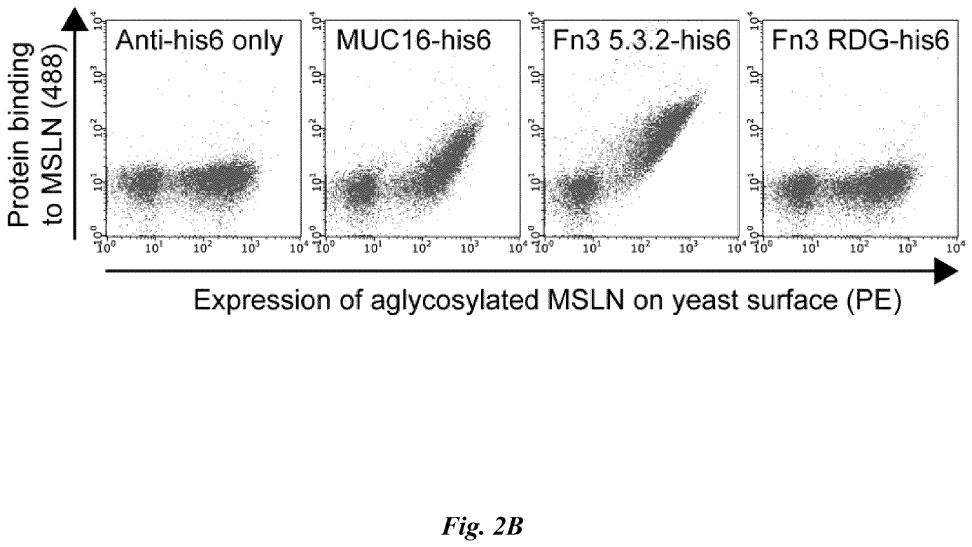

Clinical samples of PM and other MSLN-positive tumors have been shown to have variable patterns of glycans at three N-linked glycosylation sites.⁶⁹ Ideally, a theranostic recognizing MSLN would bind MSLN independent of glycosylation pattern. In addition to its application in directed evolution, yeast surface display has been used to study the interaction of ligands and antibodies with their receptors, such as to map the binding epitopes of antibodies to their receptors.⁷⁰ To determine if Fn3 5.3.2 could bind a variant of MSLN with removal of the N-linked glycans, the same MSLN sequence that was used as soluble target for sorting (Glu296-Gly580) was instead displayed on the surface of yeast, with the three asparagine residues associated with confirmed N-linked glycosylation mutated to glutamine to remove the glycosylation recognition motif. Expression of this aglycosylated MSLN was induced, and full-length expression on the yeast surface was confirmed by flow cytometry using an antibody that recognizes a C-terminal c-myc epitope tag (FIG. 2A). The distribution observed of negative and positive expressing populations is an inherent characteristic of yeast surface display, and the negative population can serve as an internal control for non-specific binding. We also confirmed that the yeast population expressing MSLN was detected by an anti-MSLN antibody (FIG. 2A).

MUC16, also known as CA125, is a native ligand for MSLN, and has previously been shown experimentally to bind an N-terminal domain of MSLN away from any MSLN glycans, mediated by glycans on MUC16.^{4,71} To confirm functional fold of displayed MSLN, we incubated saturating amounts of soluble MUC16 with a hexahistidine tag (SEQ ID NO: 33) with yeast surface-displayed MSLN. The MSLN-expressing population of yeast bound MUC16 (FIG. 2B). Soluble Fn3 5.3.2 with a hexahistidine tag (SEQ ID NO: 33) similarly bound this non-glycosylated form of MSLN (FIG. 2B). A hexahistidine-tagged (SEQ ID NO: 33) Fn3 RDG protein that is a negative control Fn3 variant that does not bind any target did not demonstrate any binding signal. The secondary antibody to the hexahistidine tag (SEQ ID NO: 33) used alone did not bind MSLN, further confirming that the binding signal was specific to ligand-MSLN interactions. We concluded that Fn3 5.3.2 binding to MSLN did not require the presence of any of the three N-linked glycans on MSLN, and we expect that glycosylation variation of MSLN among PM patients will not affect use of Fn3 5.3.2 in diagnosing or treating PM.

### Example 3: MSTO-211H Derived Clonal Populations Exhibited High Level of MSLN Expression

In pilot experiments with PM cells expressing MSLN at their natural levels, the maximum signal detected by flow cytometry for both MSLN expression and binding to Fn3 5.3.2 was low, and a larger dynamic range was needed for accurate measurements in equilibrium binding assays (data not shown). To better measure the binding affinity of Fn3 5.3.2 for MSLN, we first generated stably MSLN-overexpressing cells from the wild-type MSTO-211H cell line. We evaluated the MSLN expression on the cell surface through flow cytometry by incubating the cells with a rabbit anti-MSLN primary antibody and a goat anti-rabbit secondary PE-conjugated antibody.

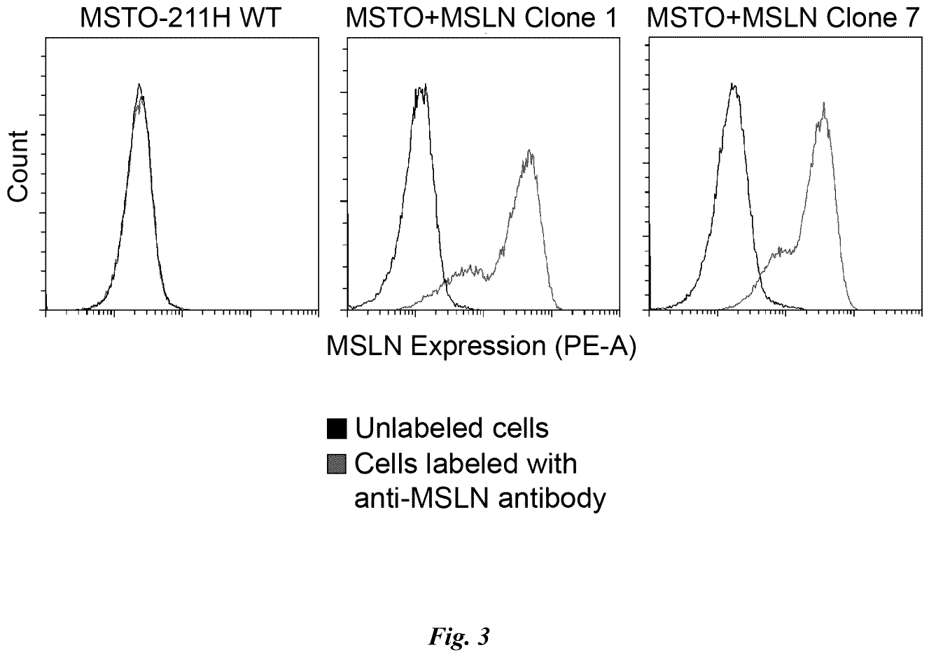

As shown in FIG. 3, no MSLN expression was detectable on the wild-type MSTO-211H cell line, as no difference was observable between the fluorescence intensity of the untreated cells (black) and that of the cells treated with the MSLN detection antibodies (red). Among the clones deriving from the clonal selection steps described in the Materials and Methods section, two clonal populations, namely “MSTO clone 1” and “MSTO clone 7”, stably expressed high MSLN levels. Specifically, the MSTO clone 1 labeled with the detection antibodies showed a 27-fold increased fluorescent signal compared with the unlabeled control cells and a 12-fold increased signal compared with the wild-type MSTO-211H. Similarly, the MSTO clone 7 incubated with the detection antibodies showed a signal increase of 17-fold compared with the unlabeled cells and 10-fold compared with the wild-type MSTO-211H. The other viable clones evaluated did not exhibit any increase in the MSLN expression and were thus not employed for subsequent analysis.

### Example 4: Fn3 5.3.2 Binds MSLN-Overexpressing MSTO Clones with High Affinity

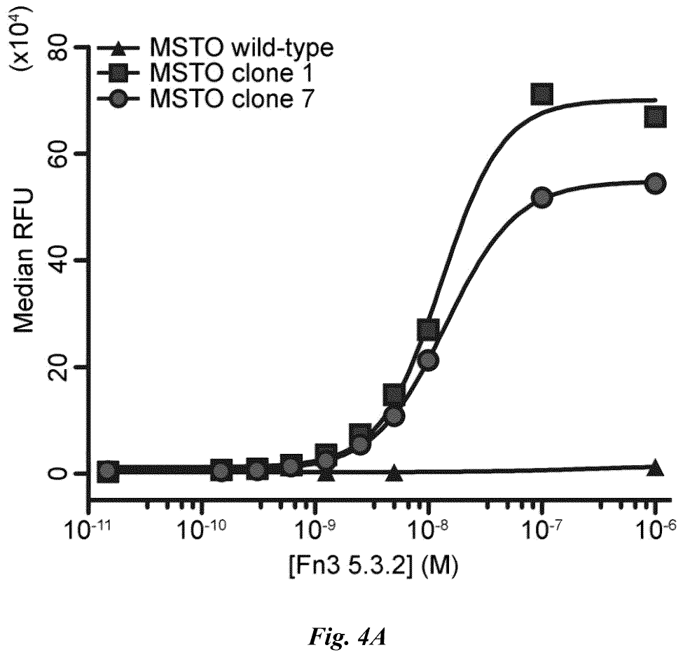

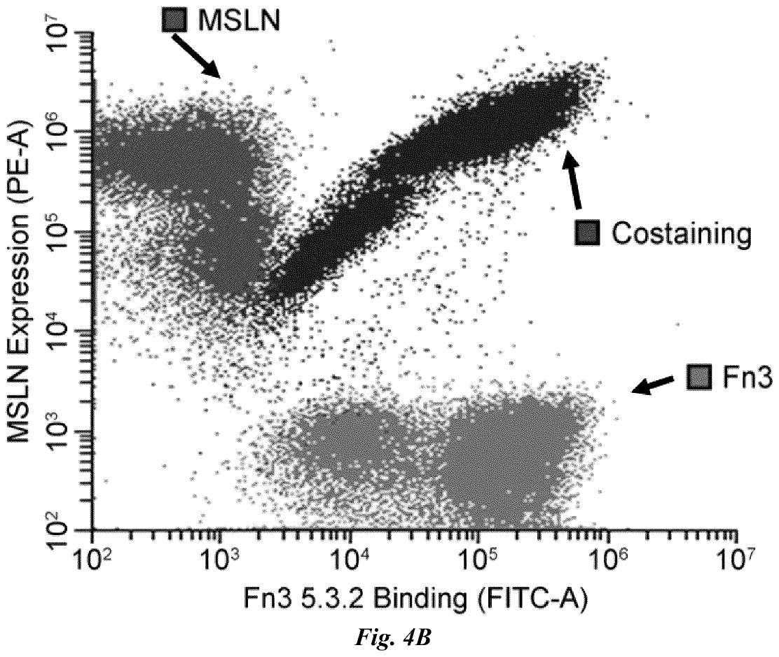

The binding affinity of Fn3 5.3.2 for MSLN was evaluated using the two MSLN-overexpressing clones, MSTO clone 1 and MSTO clone 7. Each cell line was incubated with a range of concentrations (0.015-1000 nM) of Fn3 5.3.2 with a hexahistidine tag (SEQ ID NO: 33), and the binding of Fn3 was detected through flow cytometry using an anti-HisTag antibody (FIG. 4A). The MSLN-negative cells MSTO-211H wild-type were also employed as a negative control to exclude non-specific binding between Fn3 5.3.2 and cell surface proteins other than MSLN. For MSTO clone 1 and 7, three replicates were carried out. We measured a K_{D}=11±4 nM when using MSTO clone 1, and similarly a K_{D})=11+2 nM for MSTO clone 7. Conversely, we did not observe any binding between Fn3 5.3.2 and the MSLN-negative cell line MSTO-211H wild-type. These results highlighted a specific, tight binding affinity between Fn3 5.3.2 and MSLN.

To further corroborate our observation, we incubated the MSTO clone 1 cell line with either a PE-conjugated anti-MSLN primary antibody (MSLN), Fn3 5.3.2 at a final concentration of 10 nM and the FITC-conjugated anti-HisTag antibody (Fn3), or the combination of the two treatments (costaining). Each sample was then evaluated through flow cytometry, and the fluorescence intensity detected in the PE and FITC channels was reported. The costaining sample showed a correlation between the MSLN and the Fn3 5.3.2 signal intensity that was not present in the control samples (MSLN or Fn3) (FIG. 4B). These observations highlighted that the Fn3 5.3.2 molecules binding on the cell surface were proportional to the MSLN expression, further supporting the specificity of the Fn3 MSLN binding.

### Example 5: Fn3 5.3.2 and MSLN Colocalize and Internalize into MSLN-Positive MSTO Cell Lines

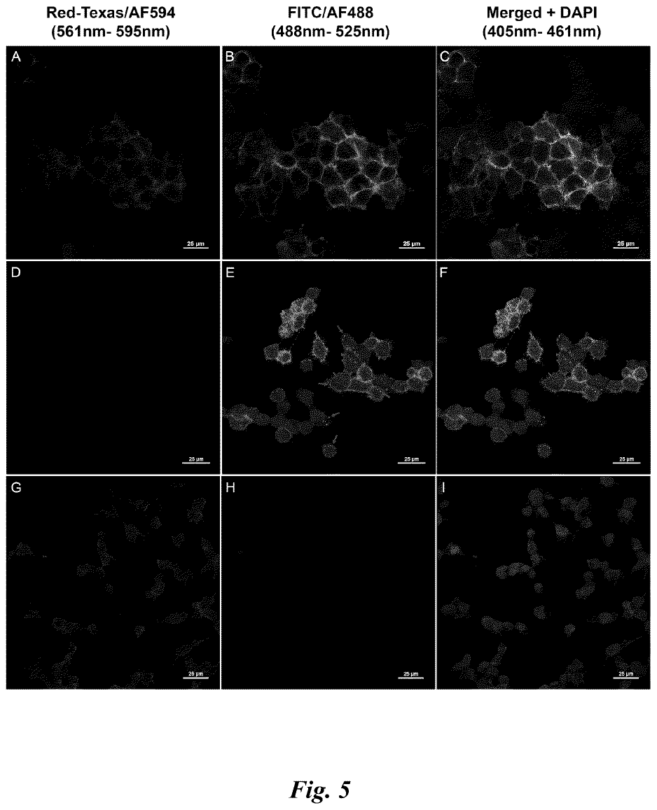

We carried out an immunofluorescence assay to visualize the localization of MSLN and Fn3 5.3.2 in the established MSLN-positive PM cell lines. MSTO-7 cells were incubated with a combination of PE-conjugated anti-MSLN primary antibody, Fn3 5.3.2 with hexahistidine tag (SEQ ID NO: 33) at a final concentration of 100 nM, and an Fn3-detecting, AF488-conjugated anti-HisTag antibody (co-staining). To study how the MSLN/Fn3 complex behaves upon binding, cells were incubated at different temperatures (4° C. or 37° C.). The fluorescence signal was detected through confocal microscopy using a Red-Texas filter for MSLN (FIG. 5, panels A, D, and G) and a FITC filter for Fn3 (FIG. 5, panels B, E, and H). In samples incubated at 4° C., staining for Fn3 5.3.2 and MSLN were colocalized on the surface of both cell lines (FIG. 5, panel C). Cells in the populations that did not express MSLN could be visualized by DAPI staining but had neither MSLN nor Fn3 5.3.2 staining, further confirming the specificity of the Fn3 5.3.2 interaction with MSLN on the tumor cell surface. In samples incubated at 37° C. for 1 h, Fn3 5.3.2 staining revealed a signal of internalization in PM cells, supporting the findings of Sirois and colleagues⁵⁸ (FIG. 5, panels E and F, red arrows). Meanwhile, after 1 h incubation, the PE-conjugated anti-MSLN antibody is mostly localized on the membrane of the cells, showing low or no internalization (FIG. 5, panels G and I). Since MSLN shedding is hypothesized to cause an “on target/off tumor” effect reducing therapeutic efficacy of antibody and CAR T-cell therapies⁶⁰, a drug carrier with rapid internalization, not limited by MSLN cleavage, could be a valuable strategy to improve the effectiveness of molecular imaging and targeted therapies. Moreover, Fn3 internalization in cancer cells paves the way for using radio-targeted alpha therapy against MSLN-positive tumors.⁶¹ Coupling Fn3 with alpha emitters could lower the damage to healthy tissues surrounding cancer cells.⁶² Pre-clinical and clinical studies have recently been reported using MSLN-targeted alpha-therapy, reporting high selectivity and efficacy in killing MSLN-positive tumor cells.^{63,64}

### Materials and Methods

### Directed Evolution and Affinity Maturation of Fn3 Variants to Bind MSLN

A hydrophilic Gr2 Fn3 library (FIG. 1A, 1B) previously evolved for MSLN-binding Fn3 variants was further affinity matured using yeast surface display and directed evolution.^{58,59,65-68} Briefly, error-prone PCR with nucleotide analogs was used to add diversity to binding loops and the overall protein framework to generate a fourth generation library (5.5×10⁷ transformants). The library was sorted twice by magnetic-activated cell-sorting (MACS) using biotinylated, Fc-tagged recombinant human MSLN (Acro Biosystems #MSN-H826x) followed by a fluorescence-activated cell sorting (FACS) selection for full-length clones using an antibody against the C-terminal c-myc epitope tag. Full-length clones were incubated with a chicken anti-c-myc antibody and the biotinylated Fc-tagged MSLN. To increase the sorting stringency, concentrations of MSLN were decreased over four iterative rounds of enrichment, with reducing concentrations of MSLN for each round to increase sort stringency. Cells were washed and incubated with a goat anti-chicken Alexa Fluor-647 (AF647) conjugate and AF488-conjugated streptavidin. Cells were washed and double-positive yeast cells were collected on a BD BioSciences FACSAria II. A sort window was created collecting 0.1-1% of cells, sorting on a diagonal by collecting those yeast that exhibited the greatest MSLN binding for a given expression level. The number of cells sorted was at least ten times the size of the present library at that sort, such that each unique variant should be sampled. Plasmid DNA from the enriched fourth generation library was recovered using a Zymoprep Yeast Plasmid Miniprep II kit (Zymo Research) following manufacturer's protocol, isolating DNA from the pooled yeast population, collecting DNA from sufficient number of cells to have 10-fold coverage of the smallest collected population during sorting, such that all unique plasmid sequences would be statistically likely to be sampled. The population DNA was transformed into XL1-Blue bacteria (5 μl of Zymoprep DNA, typical yield 1.25×10⁷ plasmids/μl, for each 50 μl of bacteria) and plated onto selective media to isolate colonies with unique plasmids. Plasmids from individual bacterial clones were sequenced by standard Sanger DNA sequencing methods. Batches of additional plasmids were sequenced until no new sequences emerged. Unique plasmids were then individually transformed into EBY100 yeast to enable binding measurements with individual protein sequences. A fifth generation library (4.4×10⁷ transformants) was generated from the enriched, pooled fourth generation DNA, and similarly sorted using MACS and FACS, with a final third FACS sort using 1 nM of biotinylated Fc-tagged MSLN. Unique plasmid sequences were determined as described for the fourth generation library.

### Affinity Measurements Using Yeast Surface Display

To identify lead Fn3 variants for soluble expression and further characterization, an equilibrium binding assay was performed with individual Fn3 variants displayed on the yeast surface and using soluble, biotinylated Fc-MSLN to measure an apparent binding affinity. These measurements have been shown to align well with binding assays using soluble protein and human cell lines, and with in vitro measurements, such as surface plasmon resonance.⁶⁷ Yeast displaying an identified Fn3 variant were incubated with a range of concentrations of biotinylated Fc-MSLN for 1 h at room temperature (RT) with gentle mixing, and with a mouse 9E10 anti-c-myc antibody to detect full-length protein expression (1:50 dilution, 0.01 mg/mL, Fisher Scientific, 13-250-0). Yeast were washed with phosphate-buffered saline (PBS) with 0.1% w/v bovine serum albumin (PBSA), and incubated with streptavidin-AF488 and with goat-anti-mouse PE-conjugated secondary antibody for 30 min at 4° C. Yeast were washed and immediately analyzed on a Guava flow cytometer (Millipore), and data were compensated for spectral overlap. Mean fluorescence intensity from binding of the expressing population was measured, plotted, and fit with a sigmoidal curve using Kaleidagraph (Synergy Software). The apparent KD was determined as the concentration of soluble Fc-MSLN yielding the half-maximal signal. At least three replicates were measured, and the mean and standard deviation were reported.

### Fn3 Expression and Purification

MSLN-binding Fn3 variant 5.3.2 was prepared as previously described.^{58,59} Briefly, Fn3 gene was cloned into a pET vector with a C-terminal hexahistidine tag (SEQ ID NO: 33) and expressed in BL21(DE3) E. coli. Cultures were grown in LB with kanamycin (50 μg/mL) and induced overnight at 20° C. with 0.5 mM isopropyl-b-D-thiogalactopyranoside (IPTG). Cells were resuspended in lysis buffer (35 mM Na₂HPO₄×dibasic, 15 mM NaH₂PO₄×monobasic, 500 mM NaCl, 5 mM CHAPS, 25 mM imidazole, 5% glycerol) supplemented with an EDTA-free protease inhibitor (Pierce), and lysed by repeated freezing and thawing. Soluble fractions were isolated by centrifugation. Fn3 variants were purified by cobalt affinity chromatography with HisPur cobalt resin (Thermo Fisher). Protein samples were dialyzed into water, lyophilized, reconstituted with 1×PBS to the desired concentration, and analyzed for purity by SDS-PAGE on a BioRad ChemiDoc MP imaging system.

Binding Experiments with MSLN Displayed on the Yeast Surface

The sequence for the full length shed MSLN used as target for directed evolution (residues Glu296-Gly580) was cloned into the yeast surface display pCT plasmid between the NheI and BamHI restriction sites following established methods.^{67,68} A gene block of the full-length gene was ordered from IDT to be used as a PCR template, with the three N-linked glycosylation sites at Asn388, Asn488, and Asn515 removed by mutation of Asn to Gln. Following subcloning and transformation into XL1-Blue E. coli, the sequence was confirmed by standard Sanger sequencing. Plasmid was transformed into EBY100 S. saccharomyces surface display strain following protocols for the Frozen-EZ Yeast Transformation II Kit (Zymo Research). An individual yeast colony with plasmid from an SD-CAA plate was grown overnight in SD-CAA liquid culture at 30° C. with aeration, then induced in SG-CAA liquid media at 20° C. with aeration for 12-24 h. Full-length expression of the MSLN on the yeast cell surface was confirmed by flow cytometry using a 9E10 antibody to the C-terminal c-myc tag (1:50 dilution) and a goat anti-mouse PE conjugate antibody (1:25 dilution). To measure binding of the rabbit anti-MSLN antibody (Abcam, EPR 19025-42), 5×10⁵ yeast displaying MSLN without N-linked glycosylation were incubated for 1 h at RT and gentle mixing with mouse 9E10 anti-c-myc antibody to measure protein expression and with rabbit anti-MSLN antibody (1:50 dilution) to measure binding in a total volume of 50 μl PBSA. Samples were washed with PBSA, then incubated for 30 min at 4° C. with a goat anti-mouse 488 conjugate (Thermo Fisher, A11001, 1:200 dilution) and a goat anti-rabbit PE conjugate (Abcam, ab72465, 1:200 dilution) in 25 μl PBSA. Samples were washed with PBSA, and immediately analyzed on a Guava flow cytometer. Measurements of MUC16, Fn3 5.3.2, or negative control Fn3 RDG binding were performed similarly. In the primary incubation, 100 nM of MUC16, Fn3 5.3.2, or Fn3 RDG was incubated with the MSLN-displaying yeast, with a chicken anti-c-myc antibody to detect full length MSLN expression. MUC16, Fn3 5.3.2, and Fn3 RDG contain a hexahistidine tag (SEQ ID NO: 33), which was detected by an anti-6×His Tag Dylight 488 antibody (“His6” disclosed as SEQ ID NO: 33) raised in mouse (Abcam, ab117512, 1:50 dilution) during the secondary incubation step, and a goat anti-chicken PE conjugate was used to detect c-myc antibody. A negative control sample of no protein ligand, but including the c-myc tag detection and both the anti-His6 (SEQ ID NO: 33) and anti-chicken secondary antibodies, was also performed. Data was analyzed in Guava InCyte software, compensated for spectral overlap of fluorescence. All binding experiments were repeated on at least three separate days.

Generation of Mesothelioma Cell Line with Enhanced MSLN Expression

MSLN-overexpressing clonal populations were established starting from the MSLN-negative cell line MSTO-211H using a pcDNA3.1 (+)-based expression vector. The vector was purchased from Twin Helix (MSLN_pcDNA3.1) and harbored the coding sequence for the MSLN transcript variant 1 (accession number: NM_005823.6) and an upstream CMV constitutive promoter. The same vector also carried the NeoR gene, which provided resistance to the G418 antibiotic. MSLN_pcDNA3.1 (5 ug) was transfected into MSTO-211H cells using Lipofectamine 3000 (Invitrogen) following manufacturer instructions. Six days after transfection, the G418 antibiotic was added and maintained in the culture medium at a final concentration of 500 μg/mL to select those cells with a stable expression of the NeoR gene. After 20 days of G418 selection, MSLN expression was evaluated. To this end, 2×10⁶ cells were harvested using a non-enzymatic solution (0.02% EDTA in PBS) and incubated with either PBS (control) or a primary rabbit anti-MSLN antibody (1:50; 9 μg/mL; #196235, Abcam) for 1 h at 4° C. The cells were then washed twice with a solution of PBSA and incubated with either PBS (control) or a secondary PE-conjugated goat anti-rabbit antibody (1:200, 2.5 ug/mL #72465, Abcam) for 30 min at 4° C. The cells were then washed twice with a solution of PBSA and incubated with either PBS (control) or a secondary PE-conjugated goat anti-rabbit antibody (1:200, #72465, Abcam) for 30 min at 4° C. Following this staining procedure, MSLN expression was evaluated through flow cytometry, and the MSLN-positive cells were seeded at a single-cell density into 96-well plates using a BD FACSJazz cell sorter. The resulting viable clonal populations were re-evaluated for their MSLN levels following a similar staining procedure, and the clones exhibiting stable MSLN overexpression were selected for evaluating Fn3 5.3.2-MSLN binding affinity.

### Cell Lines and Cell Culture

The MSTO-211H cell line was purchased from the American Type Culture Collection (ATCC, #CRL-2081). The cells were cultured in RPMI-1640 (#ECB2000L, Euroclone, S.p.A., Milan, Italy) supplemented with 10% fetal bovine serum (FBS, #10270-106, Gibco™) and 1% penicillin-streptomycin (#ECB3001D, Euroclone, S.p.A., Milan, Italy). Cells were grown at 37° C. in a humidified atmosphere with 5% CO₂.

Equilibrium Binding Assays with Cancer Cell Lines

Wild-type MSTO and MSLN-overexpressing clones (1 and 7) were detached with 0.02% EDTA (#E5134, Sigma-Aldrich) solution. For each sample, 3×10⁵ cells were pelleted at 400 g for 5 min at 4° C.; then, cells were washed with PBSA. Cells were incubated with a range of concentrations (0.015-1000 nM) of Fn3 5.3.2 in a total volume of 300 μl PBSA for 1 hour at 4° C. with rotation. Cells were washed with PBSA and incubated with a mouse anti-His6 DyLight-488 antibody (“His6” disclosed as SEQ ID NO: 33) (1:50, 20 μg/mL, #117512, Abcam) in a total volume of 50 μl for 30 min at 4° C. with rotation and protection from light. MSLN expression was detected by a rabbit anti-MSLN antibody (1:50; 9 μg/mL, #196235, Abcam) and a goat anti-rabbit PE conjugate (1:200, 2.5 μg/mL, #72465, Abcam). After incubation, cells were washed with PBSA and incubated with a mouse anti-His6 DyLight-488 antibody (“His6” disclosed as SEQ ID NO: 33) (1:50, #117512, Abcam) in a total volume of 50 μl for 30 min at 4° C. with rotation and protection from light. MSLN expression was detected by a rabbit anti-MSLN antibody (1:50; #196235, Abcam) and a goat anti-rabbit PE conjugate (1:200, #72465, Abcam). After incubation, cells were washed with PBSA and analyzed using a CytoFLEX Flow Cytometer (Beckman Coulter). Data was fit to a sigmoidal curve using a four parameter logistic (4PL) regression model. Dissociation constants (K_{D}) were calculated as the Fn3 concentration yielding half of the maximum signal for three replicates. Mean and standard deviation for the K_{D} are shown.

### Confocal Microscopy of Colocalization of Fn3 to the Membrane

MSTO and MSLN-overexpressing clones were seeded in a 6-well plate to achieve 80% confluency after 24 h, each well containing one coverslip. To study the co-localization of Fn3 and MSLN after 24 h, cells were fixed with 4% paraformaldehyde (PFA) for 15 min at RT, and washed twice with PBSA for 5 min in oscillation. Cells were blocked with 4% horse serum for 1 h at RT, and washed with PBSA. For MSLN staining, cells were treated with 50 μl PE-conjugated primary anti-MSLN antibody (1:200, 2.5 μg/mL, #ab252136 Abcam) and incubated for 1 h at 4° C. To evaluate the binding between MSLN and Fn3, cells were treated with 100 nM of Fn3 5.3.2 in a total volume of 50 μl, and incubated for 1 h at 4° C. The costaining was performed incubating cells with both treatments describe above. Then, cells were washed thrice with PBSA for 5 min in oscillation. For MSLN-Fn3 binding sample, and costaining, cells were treated with a mouse anti-His6 DyLight-488 antibody (“His6” disclosed as SEQ ID NO: 33) (1:100, 10 μg/mL, #117512, Abcam) in a total volume of 50 μl, and incubated for 1 h at 4° C. and washed as before. Cell nuclei were stained using DAPI (1:1000, ThermoFisher) and incubated for 5 min at RT protected from light and washed twice with PBSA. The control sample was incubated with a mouse anti-His6 DyLight-488 antibody (“His6” disclosed as SEQ ID NO: 33) (1:100, 10 μg/mL, #117512, Abcam) without Fn3, under the same conditions. Coverslips were mounted on microscope slides with mounting medium (#F4680, Sigma-Aldrich) and sealed with nail polish. Samples were observed using a Nikon A1+confocal microscope.

### Confocal Microscopy of Internalization of Fn3/MSLN in Cancer Cells

To study the internalization of MSLN/Fn3 5.3.2 complex into PM cancer cells, MSTO clone 7 was treated as described before. Briefly, MSTO clone 7 cells were seeded in a 6-well containing one coverslip. When the cells reached 80% confluence (after 48 hours), cells were treated with PE-conjugated primary anti-MSLN antibody (1:200, 2.5 g/mL, #ab252136 Abcam) or with 100 nM of Fn3 5.3.2 in a total volume of 50 μl, and incubated for 1 h at 37° C. After a wash with PBSA for 5 min in oscillation, cells were fixed with 4% paraformaldehyde (PFA) for 15 min at RT, and washed twice with PBSA for 5 min in oscillation. To ensure permeabilization, cells were incubated with 1 mL Triton 0.25% for 5 min at RT and washed twice with PBSA while oscillating. The blocking step with HS 4%, incubation with anti-His6 DyLight-488 antibody (“His6” disclosed as SEQ ID NO: 33) (1:100,10 ug/mL, #117512, Abcam) and nuclei staining with DAPI were carried out as described in the previous section. Coverslips were mounted on microscope slides as described previously. Samples were analysed using a Nikon TI2 confocal microscope. To evaluate the spatial arrangement of MSLN/Fn3 internalization, z-stack imaging was used (0.2 um steps, range ˜10 um).

### ABBREVIATIONS

AF-Alexa Fluor
DAPI-4′,6-diamidino-2-phenylindole
FACS-fluorescence activated cell sorting
FITC-fluorescein isothiocyanate
Fn3-human fibronectin type III tenth domain
IPTG-isopropyl-b-D-thiogalactopyranoside
MACS-magnetic activated cell sorting
MSLN-mesothelin
MW-molecular weight
PBS-phosphate buffered saline
PBSA-phosphate buffered saline, with 0.1% (w/v) bovine serum albumin
PE-phycoerythrin
PFA-paraformaldehyde
PM-pleural mesothelioma

### REFERENCES

(1) Bera, T. K.; Pastan, I. Mesothelin Is Not Required for Normal Mouse Development or Reproduction. Mol Cell Biol 2000, 20 (8), 2902-2906. doi.org/10.1128/MCB.20.8.2902-2906.2000.
(2) Yeo, D.; Castelletti, L.; van Zandwijk, N.; Rasko, J. E. J. Hitting the Bull's-Eye: Mesothelin's Role as a Biomarker and Therapeutic Target for Malignant Pleural Mesothelioma. Cancers (Basel) 2021, 13 (16), 3932. doi.org/10.3390/cancers13163932.
(3) Rump, A.; Morikawa, Y.; Tanaka, M.; Minami, S.; Umesaki, N.; Takeuchi, M.; Miyajima, A. Binding of Ovarian Cancer Antigen CA125/MUC16 to Mesothelin Mediates Cell Adhesion. J Biol Chem 2004, 279 (10), 9190-9198. doi.org/10.1074/jbc.M312372200.
(4) Gubbels, J. A.; Belisle, J.; Onda, M.; Rancourt, C.; Migneault, M.; Ho, M.; Bera, T. K.; Connor, J.; Sathyanarayana, B. K.; Lee, B.; Pastan, I.; Patankar, M. S. Mesothelin-MUC16 Binding Is a High Affinity, N-Glycan Dependent Interaction That Facilitates Peritoneal Metastasis of Ovarian Tumors. Mol Cancer 2006, 5 (1), 50. doi.org/10.1186/1476-4598-5-50.
(5) Weidemann, S.; Gagelmann, P.; Gorbokon, N.; Lennartz, M.; Menz, A.; Luebke, A. M.; Kluth, M.; Hube-Magg, C.; Blessin, N. C.; Fraune, C.; Möller, K.; Bernreuther, C.; Lebok, P.; Clauditz, T. S.; Jacobsen, F.; Izbicki, J. R.; Jansen, K.; Sauter, G.; Uhlig, R.; Wilczak, W.; Steurer, S.; Minner, S.; Burandt, E.; Krech, R. H.; Dum, D.; Krech, T.; Marx, A. H.; Simon, R. Mesothelin Expression in Human Tumors: A Tissue Microarray Study on 12,679 Tumors. Biomedicines 2021, 9 (4), 397. doi.org/10.3390/biomedicines9040397.
(6) Carbone, M.; Adusumilli, P. S.; Alexander, H. R.; Baas, P.; Bardelli, F.; Bononi, A.; Bueno, R.; Felley-Bosco, E.; Galateau-Salle, F.; Jablons, D.; Mansfield, A. S.; Minaai, M.; de Perrot, M.; Pesavento, P.; Rusch, V.; Severson, D. T.; Taioli, E.; Tsao, A.; Woodard, G.; Yang, H.; Zauderer, M. G.; Pass, H. I. Mesothelioma: Scientific Clues for Prevention, Diagnosis, and Therapy. CA Cancer J Clin 2019, 69 (5), 402-429. doi.org/10.3322/caac.21572.
(7) Sung, H.; Ferlay, J.; Siegel, R. L.; Laversanne, M.; Soerjomataram, I.; Jemal, A.; Bray, F. Global Cancer Statistics 2020: GLOBOCAN Estimates of Incidence and Mortality Worldwide for 36 Cancers in 185 Countries. CA Cancer J Clin 2021, 71 (3), 209-249. doi.org/10.3322/caac.21660.
(8) Bronte, G.; Incorvaia, L.; Rizzo, S.; Passiglia, F.; Galvano, A.; Rizzo, F.; Rolfo, C.; Fanale, D.; Listì, A.; Natoli, C.; Bazan, V.; Russo, A. The Resistance Related to Targeted Therapy in Malignant Pleural Mesothelioma: Why Has Not the Target Been Hit Yet? Crit Rev Oncol Hematol 2016, 107, 20-32. doi.org/10.1016/j.critrevonc.2016.08.011.
(9) Danlos, F.-X.; Texier, M.; Job, B.; Mouraud, S.; Cassard, L.; Baldini, C.; Varga, A.; Yurchenko, A. A.; Rabeau, A.; Champiat, S.; Letourneur, D.; Bredel, D.; Susini, S.; Blum, Y.; Parpaleix, A.; Parlavecchio, C.; Tselikas, L.; Fahrner, J.-E.; Goubet, A.-G.; Rouanne, M.; Rafie, S.; Abbassi, A.; Kasraoui, I.; Breckler, M.; Farhane, S.; Ammari, S.; Laghouati, S.; Gazzah, A.; Lacroix, L.; Besse, B.; Droin, N.; Deloger, M.; Cotteret, S.; Adam, J.; Zitvogel, L.; Nikolaev, S. I.; Chaput, N.; Massard, C.; Soria, J.-C.; Gomez-Roca, C.; Zalcman, G.; Planchard, D.; Marabelle, A. Genomic Instability and Protumoral Inflammation Are Associated with Primary Resistance to Anti-PD-1+Antiangiogenesis in Malignant Pleural Mesothelioma. Cancer Discov 2023, 13 (4), 858-879. doi.org/10.1158/2159-8290.CD-22-0886.
(10) Nicolini, F.; Bocchini, M.; Bronte, G.; Delmonte, A.; Guidoboni, M.; Crinò, L.; Mazza, M. Malignant Pleural Mesothelioma: State-of-the-Art on Current Therapies and Promises for the Future. Front Oncol 2019, 9, 1519. doi.org/10.3389/fonc.2019.01519.
(11) Melaiu, O.; Melissari, E.; Mutti, L.; Bracci, E.; De Santi, C.; Iofrida, C.; Di Russo,
M.; Cristaudo, A.; Bonotti, A.; Cipollini, M.; Garritano, S. I.; Foddis, R.; Lucchi, M.; Pellegrini, S.; Gemignani, F.; Landi, S. Expression Status of Candidate Genes in Mesothelioma Tissues and Cell Lines. Mutation Research Fundamental and Molecular Mechanisms of Mutagenesis 2015, 771, 6-12. doi.org/10.1016/j.mrfmmm.2014.11.002.
(12) Borea, F.; Franczak, M. A.; Garcia, M.; Perrino, M.; Cordua, N.; Smolenski, R. T.; Peters, G. J.; Dziadziuszko, R.; Santoro, A.; Zucali, P. A.; Giovannetti, E. Target Therapy in Malignant Pleural Mesothelioma: Hope or Mirage? Int J Mol Sci 2023, 24(11), 9165. doi.org/10.3390/ijms24119165.
(13) Kindler, H. L.; Novello, S.; Bearz, A.; Ceresoli, G. L.; Aerts, J. G. J. V.; Spicer, J.; Taylor, P.; Nackaerts, K.; Greystoke, A.; Jennens, R.; Calabrò, L.; Burgers, J. A.; Santoro, A.; Cedres, S.; Serwatowski, P.; Ponce, S.; Van Meerbeeck, J. P.; Nowak, A. K.; Blumenschein, G.; Siegel, J. M.; Kasten, L.; Köchert, K.; Walter, A. O.; Childs, B. H.; Elbi, C.; Hassan, R.; Fennell, D. A. Anetumab Ravtansine versus Vinorelbine in Patients with Relapsed, Mesothelin-Positive Malignant Pleural Mesothelioma (ARCS-M): A Randomised, Open-Label Phase 2 Trial. The Lancet Oncology 2022, 23 (4), 540-552. doi.org/10.1016/S1470-2045 (22) 00061-4.
(14) Hassan, R.; Kindler, H. L.; Jahan, T.; Bazhenova, L.; Reck, M.; Thomas, A.; Pastan, I.; Parno, J.; O'Shannessy, D. J.; Fatato, P.; Maltzman, J. D.; Wallin, B. A. Phase II Clinical Trial of Amatuximab, a Chimeric Antimesothelin Antibody with Pemetrexed and Cisplatin in Advanced Unresectable Pleural Mesothelioma. Clin Cancer Res 2014, 20 (23), 5927-5936. doi.org/10.1158/1078-0432.CCR-14-0804.
(15) Fujisaka, Y.; Kurata, T.; Tanaka, K.; Kudo, T.; Okamoto, K.; Tsurutani, J.; Kaneda, H.; Okamoto, I.; Namiki, M.; Kitamura, C.; Nakagawa, K. Phase I Study of Amatuximab, a Novel Monoclonal Antibody to Mesothelin, in Japanese Patients with Advanced Solid Tumors. Invest New Drugs 2015, 33 (2), 380-388. doi.org/10.1007/s10637-014-0196-0.
(16) Matsuzawa, F.; Kamachi, H.; Mizukami, T.; Einama, T.; Kawamata, F.; Fujii, Y.; Fukai, M.; Kobayashi, N.; Hatanaka, Y.; Taketomi, A. Mesothelin Blockage by Amatuximab Suppresses Cell Invasiveness, Enhances Gemcitabine Sensitivity and Regulates Cancer Cell Stemness in Mesothelin-Positive Pancreatic Cancer Cells. BMC Cancer 2021, 21 (1), 200. doi.org/10.1186/s12885-020-07722-3.
(17) Mizukami, T.; Kamachi, H.; Fujii, Y.; Matsuzawa, F.; Einama, T.; Kawamata, F.; Kobayashi, N.; Hatanaka, Y.; Taketomi, A. The Anti-Mesothelin Monoclonal Antibody Amatuximab Enhances the Anti-Tumor Effect of Gemcitabine against Mesothelin-High Expressing Pancreatic Cancer Cells in a Peritoneal Metastasis Mouse Model. Oncotarget 2018, 9 (73), 33844-33852. doi.org/10.18632/oncotarget.26117.
(18) Nicolaides, N. C.; Schweizer, C.; Somers, E. B.; Wang, W.; Fernando, S.; Ross, E. N.; Grasso, L.; Hassan, R.; Kline, J. B. CA125 Suppresses Amatuximab Immune-Effector Function and Elevated Serum Levels Are Associated with Reduced Clinical Response in First Line Mesothelioma Patients. Cancer Biol Ther 2018, 19 (7), 622-630. doi.org/10.1080/15384047.2018.1449614.
(19) Fujii, Y.; Kamachi, H.; Matsuzawa, F.; Mizukami, T.; Kobayashi, N.; Fukai, M.; Taketomi, A. Early Administration of Amatuximab, a Chimeric High-Affinity Anti-Mesothelin Monoclonal Antibody, Suppresses Liver Metastasis of Mesothelin-Expressing Pancreatic Cancer Cells and Enhances Gemcitabine Sensitivity in a Xenograft Mouse Model. Invest New Drugs 2021, 39 (5), 1256-1266. doi.org/10.1007/s10637-021-01118-1.
(20) Adusumilli, P. S.; Zauderer, M. G.; Rivière, I.; Solomon, S. B.; Rusch, V. W.; O'Cearbhaill, R. E.; Zhu, A.; Cheema, W.; Chintala, N. K.; Halton, E.; Pineda, J.; Perez-Johnston, R.; Tan, K. S.; Daly, B.; Araujo Filho, J. A.; Ngai, D.; McGee, E.; Vincent, A.; Diamonte, C.; Sauter, J. L.; Modi, S.; Sikder, D.; Senechal, B.; Wang, X.; Travis, W. D.; Gönen, M.; Rudin, C. M.; Brentjens, R. J.; Jones, D. R.; Sadelain, M. A Phase I Trial of Regional Mesothelin-Targeted CAR T-Cell Therapy in Patients with Malignant Pleural Disease, in Combination with the Anti-PD-1 Agent Pembrolizumab. Cancer Discov 2021, 11 (11), 2748-2763. doi.org/10.1158/2159-8290.CD-21-0407.
(21) Castelletti, L.; Yeo, D.; van Zandwijk, N.; Rasko, J. E. J. Anti-Mesothelin CAR T Cell Therapy for Malignant Mesothelioma. Biomark Res 2021, 9 (1), 11. doi.org/10.1186/s40364-021-00264-1.
(22) Pang, N.; Shi, J.; Qin, L.; Chen, A.; Tang, Y.; Yang, H.; Huang, Y.; Wu, Q.; Li, X.; He, B.; Li, T.; Liang, B.; Zhang, J.; Cao, B.; Liu, M.; Feng, Y.; Ye, X.; Chen, X.; Wang, L.; Tian, Y.; Li, H.; Li, J.; Hu, H.; He, J.; Hu, Y.; Zhi, C.; Tang, Z.; Gong, Y.; Xu, F.; Xu, L.; Fan, W.; Zhao, M.; Chen, D.; Lian, H.; Yang, L.; Li, P.; Zhang, Z. IL-7 and CCL19-Secreting CAR-T Cell Therapy for Tumors with Positive Glypican-3 or Mesothelin. J Hematol Oncol 2021, 14 (1), 118. doi.org/10.1186/s13045-021-01128-9.
(23) Schoutrop, E.; El-Serafi, I.; Poiret, T.; Zhao, Y.; Gultekin, O.; He, R.; Moyano-Galceran, L.; Carlson, J. W.; Lehti, K.; Hassan, M.; Magalhaes, I.; Mattsson, J. Mesothelin-Specific CAR T Cells Target Ovarian Cancer. Cancer Res 2021, 81 (11), 3022-3035. doi.org/10.1158/0008-5472.CAN-20-2701.
(24) Zhang, Y.; Chertov, O.; Zhang, J.; Hassan, R.; Pastan, I. Cytotoxic Activity of Immunotoxin SS1P Is Modulated by TACE-Dependent Mesothelin Shedding. Cancer Research 2011, 71 (17), 5915-5922. doi.org/10.1158/0008-5472.CAN-11-0466.
(25) Liu, X.; Chan, A.; Tai, C.-H.; Andresson, T.; Pastan, I. Multiple Proteases Are Involved in Mesothelin Shedding by Cancer Cells. Commun Biol 2020, 3 (1), 728. doi.org/10.1038/s42003-020-01464-5.
(26) Liu, X.; Onda, M.; Watson, N.; Hassan, R.; Ho, M.; Bera, T. K.; Wei, J.; Chakraborty, A.; Beers, R.; Zhou, Q.; Shajahan, A.; Azadi, P.; Zhan, J.; Xia, D.; Pastan, I. Highly Active CAR T Cells That Bind to a Juxtamembrane Region of Mesothelin and Are Not Blocked by Shed Mesothelin. Proc. Natl. Acad. Sci. U.S.A. 2022, 119 (19), e2202439119. doi.org/10.1073/pnas.2202439119.
(27) Chakraborty, A.; Onda, M.; O'Shea, T.; Wei, J.; Liu, X.; Bera, T. K.; Pastan, I. A Bispecific Antibody That Targets the Membrane-Proximal Region of Mesothelin and Retains High Anticancer Activity in the Presence of Shed Mesothelin. Molecular Cancer Therapeutics 2024, 23 (7), 1021-1030. doi.org/10.1158/1535-7163.MCT-23-0233.
(28) Lindenberg, L.; Thomas, A.; Adler, S.; Mena, E.; Kurdziel, K.; Maltzman, J.; Wallin, B.; Hoffman, K.; Pastan, I.; Paik, C. H.; Choyke, P.; Hassan, R. Safety and Biodistribution of 111 In-Amatuximab in Patients with Mesothelin Expressing Cancers Using Single Photon Emission Computed Tomography-Computed Tomography (SPECT-CT) Imaging. Oncotarget 2015, 6 (6), 4496-4504. doi.org/10.18632/oncotarget.2883.
(29) Lamberts, L. E.; Menke-van Der Houven Van Oordt, C. W.; Ter Weele, E. J.; Bensch, F.; Smeenk, M. M.; Voortman, J.; Hoekstra, O. S.; Williams, S. P.; Fine, B. M.; Maslyar, D.; De Jong, J. R.; Gietema, J. A.; Schröder, C. P.; Bongaerts, A. H. H.; Lub-de Hooge, M. N.; Verheul, H. M. W.; Sanabria Bohorquez, S. M.; Glaudemans, A. W. J. M.; De Vries, E. G. E. ImmunoPET with Anti-Mesothelin Antibody in Patients with Pancreatic and Ovarian Cancer before Anti-Mesothelin Antibody-Drug Conjugate Treatment. Clinical Cancer Research 2016, 22 (7), 1642-1652. doi.org/10.1158/1078-0432.CCR-15-1272.
(30) Conte, M.; Frantellizzi, V.; Matto, A.; De Vincentis, G. New Insight and Future Perspective of Mesothelin-Targeted Agents in Nuclear Medicine. Clin Transl Imaging 2020, 8 (4), 265-278. doi.org/10.1007/s40336-020-00379-9.
(31) Broer, L. N.; Knapen, D. G.; Suurs, F. V.; Moen, I.; Giesen, D.; Waaijer, S. J. H.; Indrevoll, B.; Ellingsen, C.; Kristian, A.; Cuthbertson, A. S.; De Groot, D.-J. A.; Cole, P. E.; De Vries, E. G. E.; Hagemann, U. B.; Lub-De Hooge, M. N. ⁸⁹ Zr-3,2-HOPO-Mesothelin Antibody PET Imaging Reflects Tumor Uptake of Mesothelin Targeted 227 Th-Conjugate Therapy in Mice. J Nucl Med 2022, jnumed. 121.263079. doi.org/10.2967/jnumed. 121.263079.
(32) Prantner, A. M.; Yin, C.; Kamat, KSharma, K.; Lowenthal, A. C.; Madrid, P. B.; Scholler, N. Molecular Imaging of Mesothelin-Expressing Ovarian Cancer with a Human and Mouse Cross-Reactive Nanobody. Mol. Pharmaceutics 2018, 15 (4), 1403-1411. doi.org/10.1021/acs.molpharmaceut.7b00789.
(33) Benloucif, A.; Meyer, D.; Balasse, L.; Goubard, A.; Danner, L.; Bouhlel, A.; Castellano, R.; Guillet, B.; Chames, P.; Kerfelec, B. Rapid Nanobody-Based Imaging of Mesothelin Expressing Malignancies Compatible with Blocking Therapeutic Antibodies. Front. Immunol. 2023, 14, 1200652. doi.org/10.3389/fimmu.2023.1200652.
(34) Tsai, W. K.; Wu, A. M. Aligning Physics and Physiology: E Ngineering Antibodies for Radionuclide Delivery. Labelled Comp Radiopharmac 2018, 61 (9), 693-714. doi.org/10.1002/jlcr.3622.
(35) Stern, L. A.; Case, B. A.; Hackel, B. J. Alternative Non-Antibody Protein Scaffolds for Molecular Imaging of Cancer. Current Opinion in Chemical Engineering 2013, 2 (4), 425-432. doi.org/10.1016/j.coche.2013.08.009.
(36) Kimura, R. H.; Cheng, Z.; Gambhir, S. S.; Cochran, J. R. Engineered Knottin Peptides: A New Class of Agents for Imaging Integrin Expression in Living Subjects. Cancer Research 2009, 69 (6), 2435-2442. doi.org/10.1158/0008-5472.CAN-08-2495.
(37) Tolmachev, V.; Tran, T. A.; Rosik, D.; Sjöberg, A.; Abrahmsén, L.; Orlova, A. Tumor Targeting Using Affibody Molecules: Interplay of Affinity, Target Expression Level, and Binding Site Composition. J Nucl Med 2012, 53 (6), 953-960. doi.org/10.2967/jnumed.111.101527.
(38) Zhang, L.; Bhatnagar, S.; Deschenes, E.; Thurber, G. M. Mechanistic and Quantitative Insight into Cell Surface Targeted Molecular Imaging Agent Design. Sci Rep 2016, 6 (1), 25424. doi.org/10.1038/srep25424.
(39) Kruziki, M. A.; Case, B. A.; Chan, J. Y.; Zudock, E. J.; Woldring, D. R.; Yee, D.; Hackel, B. J. ⁶⁴ Cu-Labeled Gp2 Domain for PET Imaging of Epidermal Growth Factor Receptor. Mol. Pharmaceutics 2016, 13 (11), 3747-3755. doi.org/10.1021/acs.molpharmaceut.6b00538.
(40) Niemeijer, A. N.; Leung, D.; Huisman, M. C.; Bahce, I.; Hoekstra, O. S.; Van Dongen, G. A. M. S.; Boellaard, R.; Du, S.; Hayes, W.; Smith, R.; Windhorst, A. D.; Hendrikse, N. H.; Poot, A.; Vugts, D. J.; Thunnissen, E.; Morin, P.; Lipovsek, D.; Donnelly, D. J.; Bonacorsi, S. J.; Velasquez, L. M.; De Gruijl, T. D.; Smit, E. F.; De Langen, A. J. Whole Body PD-1 and PD-L1 Positron Emission Tomography in Patients with Non-Small-Cell Lung Cancer. Nat Commun 2018, 9 (1), 4664. doi.org/10.1038/s41467-018-07131-y.
(41) Nessler, I.; Khera, E.; Vance, S.; Kopp, A.; Qiu, Q.; Keating, T. A.; Abu-Yousif, A. O.; Sandal, T.; Legg, J.; Thompson, L.; Goodwin, N.; Thurber, G. M. Increased Tumor Penetration of Single-Domain Antibody-Drug Conjugates Improves In Vivo Efficacy in Prostate Cancer Models. Cancer Research 2020, 80 (6), 1268-1278. doi.org/10.1158/0008-5472.CAN-19-2295.
(42) Thurber, G. M.; Schmidt, M. M.; Wittrup, K. D. Antibody Tumor Penetration: Transport Opposed by Systemic and Antigen-Mediated Clearance. Advanced Drug Delivery Reviews 2008, 60 (12), 1421-1434. doi.org/10.1016/j.addr.2008.04.012.
(43) Deonarain, M. P.; Xue, Q. Tackling Solid Tumour Therapy with Small-Format Drug Conjugates. Antibody Therapeutics 2020, 3 (4), 237-245. doi.org/10.1093/abt/tbaa024.
(44) Koide, A.; Bailey, C. W.; Huang, X.; Koide, S. The Fibronectin Type III Domain as a Scaffold for Novel Binding Proteins. Journal of Molecular Biology 1998, 284 (4), 1141-1151. doi.org/10.1006/jmbi.1998.2238.
(45) Rönnmark, J.; Grönlund, H.; Uhlen, M.; Nygren, P.-Å. Human Immunoglobulin A (IgA)-Specific Ligands from Combinatorial Engineering of Protein A: IgA-Specific Affibody Ligands. European Journal of Biochemistry 2002, 269 (11), 2647-2655. doi.org/10.1046/j.1432-1033.2002.02926.x.
(46) Kruziki, M. A.; Bhatnagar, S.; Woldring, D. R.; Duong, V. T.; Hackel, B. J. A 45-Amino-Acid Scaffold Mined from the PDB for High-Affinity Ligand Engineering. Chemistry & Biology 2015, 22 (7), 946-956. doi.org/10.1016/j.chembiol.2015.06.012.
(47) Ji, F.; Ren, J.; Vincke, C.; Jia, L.; Muyldermans, S. Nanobodies: From Serendipitous Discovery of Heavy Chain-Only Antibodies in Camelids to a Wide Range of Useful Applications. In Single-Domain Antibodies; Hussack, G., Henry, K. A., Eds.; Methods in Molecular Biology; Springer US: New York, NY, 2022; Vol. 2446, pp 3-17. doi.org/10.1007/978-1-0716-2075-5_1.
(48) Pluckthun, A. Designed Ankyrin Repeat Proteins (DARPins): Binding Proteins for Research, Diagnostics, and Therapy. Annu. Rev. Pharmacol. Toxicol. 2015, 55 (1), 489-511. doi.org/10.1146/annurev-pharmtox-010611-134654.
(49) Moore, S. J.; Cochran, J. R. Engineering Knottins as Novel Binding Agents. In Methods in Enzymology; Elsevier, 2012; Vol. 503, pp 223-251. doi.org/10.1016/B978-O-12-396962-0.00009-4.
(50) Gebauer, M.; Skerra, A. Engineered Protein Scaffolds as Next-Generation Therapeutics. Annu. Rev. Pharmacol. Toxicol. 2020, 60 (1), 391-415. doi.org/10.1146/annurev-pharmtox-010818-021118.
(51) Zahnd, C.; Kawe, M.; Stumpp, M. T.; De Pasquale, C.; Tamaskovic, R.; Nagy-Davidescu, G.; Dreier, B.; Schibli, R.; Binz, H. K.; Waibel, R.; Plückthun, A. Efficient Tumor Targeting with High-Affinity Designed Ankyrin Repeat Proteins: Effects of Affinity and Molecular Size. Cancer Research 2010, 70 (4), 1595-1605. doi.org/10.1158/0008-5472.CAN-09-2724.
(52) Moore, S. J.; Hayden Gephart, M. G.; Bergen, J. M.; Su, Y. S.; Rayburn, H.; Scott, M. P.; Cochran, J. R. Engineered Knottin Peptide Enables Noninvasive Optical Imaging of Intracranial Medulloblastoma. Proc. Natl. Acad. Sci. U.S.A. 2013, 110 (36), 14598-14603. doi.org/10.1073/pnas. 1311333110.
(53) Strohl, W. R. Fusion Proteins for Half-Life Extension of Biologics as a Strategy to Make Biobetters. BioDrugs 2015, 29 (4), 215-239. doi.org/10.1007/s40259-015-0133-6.
(54) van Witteloostuijn, S. B.; Pedersen, S. L.; Jensen, K. J. Half-Life Extension of Biopharmaceuticals Using Chemical Methods: Alternatives to PEGylation. ChemMedChem 2016, 11 (22), 2474-2495. doi.org/10.1002/cmdc.201600374.
(55) Currier, N. V.; Ackerman, S. E.; Kintzing, J. R.; Chen, R.; Filsinger Interrante, M.; Steiner, A.; Sato, A. K.; Cochran, J. R. Targeted Drug Delivery with an Integrin-Binding Knottin-Fc-MMAF Conjugate Produced by Cell-Free Protein Synthesis. Molecular Cancer Therapeutics 2016, 15 (6), 1291-1300. doi.org/10.1158/1535-7163.MCT-15-0881.
(56) Brandl, F.; Busslinger, S.; Zangemeister-Wittke, U.; Plückthun, A. Optimizing the Anti-Tumor Efficacy of Protein-Drug Conjugates by Engineering the Molecular Size and Half-Life. Journal of Controlled Release 2020, 327, 186-197. doi.org/10.1016/j.jconrel.2020.08.004.
(57) Chandler, P. G.; Buckle, A. M. Development and Differentiation in Monobodies Based on the Fibronectin Type 3 Domain. Cells 2020, 9 (3), 610. doi.org/10.3390/cells9030610.
(58) Sirois, A. R.; Deny, D. A.; Baierl, S. R.; George, K. S.; Moore, S. J. Fn3 Proteins Engineered to Recognize Tumor Biomarker Mesothelin Internalize upon Binding. PLOS ONE 2018, 13 (5), e0197029. doi.org/10.1371/journal.pone.0197029.
(59) Sirois, A. R.; Deny, D. A.; Li, Y.; Fall, Y. D.; Moore, S. J. Engineered Fn3 Protein Has Targeted Therapeutic Effect on Mesothelin-expressing Cancer Cells and Increases Tumor Cell Sensitivity to Chemotherapy. Biotechnology and Bioengineering 2020, 117 (2), 330-341. doi.org/10.1002/bit.27204.
(60) Liu X F, Onda M, Schlomer J, Bassel L, Kozlov S, Tai C-H, Zhou Q, Liu W, Tsao H-E, Hassan R, et al. (2024) Tumor resistance to anti-mesothelin CAR-T cells caused by binding to shed mesothelin is overcome by targeting a juxtamembrane epitope. Proceedings of the National Academy of Sciences 121:e2317283121.
(61) Coll R P, Bright S J, Martinus DKJ, Georgiou D K, Sawakuchi G O, Manning H C (2023) Alpha Particle-Emitting Radiopharmaceuticals as Cancer Therapy: Biological Basis, Current Status, and Future Outlook for Therapeutics Discovery. Mol Imaging Biol 25:991-1019.
(62) Sudo H, Tsuji A B, Sugyo A, Kaneko M K, Kato Y, Nagatsu K, Suzuki H, Higashi T (2021) Preclinical Evaluation of Podoplanin-Targeted Alpha-Radioimmunotherapy with the Novel Antibody NZ-16 for Malignant Mesothelioma. Cells 10:2503.
(63) Hagemann U B, Ellingsen C, Schuhmacher J, Kristian A, Mobergslien A, Cruciani V, Wickstroem K, Schatz C A, Kneip C, Golfier S, et al. (2019) Mesothelin-Targeted Thorium-227 Conjugate (MSLN-TTC): Preclinical Evaluation of a New Targeted Alpha Therapy for Mesothelin-Positive Cancers. Clin Cancer Res 25:4723-4734.
(64) Wang X, Ma W, Liu W, Ma H, Yang Y, Wang Y, Liu N, Yang G (2020) Construction and Preclinical Evaluation of 211At Labeled Anti-mesothelin Antibodies as Potential Targeted Alpha Therapy Drugs. J Radiat Res 61:684-690.
(65) Woldring, D. R.; Holec, P. V.; Zhou, H.; Hackel, B. J. High-Throughput Ligand Discovery Reveals a Sitewise Gradient of Diversity in Broadly Evolved Hydrophilic Fibronectin Domains. PLoS ONE 2015, 10 (9), e0138956. doi.org/10.1371/journal.pone.0138956.
(66) Boder, E. T.; Wittrup, K. D. Yeast Surface Display for Screening Combinatorial Polypeptide Libraries. Nat Biotechnol 1997, 15 (6), 553-557. doi.org/10.1038/nbt0697-553.
(67) Gai, S. A.; Wittrup, K. D. Yeast Surface Display for Protein Engineering and Characterization. Current Opinion in Structural Biology 2007, 17 (4), 467-473. doi.org/10.1016/j.sbi.2007.08.012.
(68) Chen, T. F.; De Picciotto, S.; Hackel, B. J.; Wittrup, K. D. Engineering Fibronectin-Based Binding Proteins by Yeast Surface Display. In Methods in Enzymology; Elsevier, 2013; Vol. 523, pp 303-326. doi.org/10.1016/B978-O-12-394292-0.00014-X.
(69) Zhan, J.; Lin, D.; Watson, N.; Esser, L.; Tang, W. K.; Zhang, A.; Liu, X.; Hassan, R.;
Gleinich, A.; Shajahan, A.; Azadi, P.; Pastan, I.; Xia, D. Structures of Cancer Antigen Mesothelin and Its Complexes with Therapeutic Antibodies. Cancer Research Communications 2023, 3 (2), 175-191. doi.org/10.1158/2767-9764.CRC-22-0306.
(70) Cochran, J. R.; Kim, Y.-S.; Olsen, M. J.; Bhandari, R.; Wittrup, K. D. Domain-Level Antibody Epitope Mapping through Yeast Surface Display of Epidermal Growth Factor Receptor Fragments. Journal of Immunological Methods 2004, 287 (1-2), 147-158. doi.org/10.1016/j.jim.2004.01.024.
(71) Kaneko, O.; Gong, L.; Zhang, J.; Hansen, J. K.; Hassan, R.; Lee, B.; Ho, M. A Binding Domain on Mesothelin for CA125/MUC16. Journal of Biological Chemistry 2009, 284 (6), 3739-3749. doi.org/10.1074/jbc.M806776200.

The publications (including patent publications), web sites, company names, books, manuals, treatise, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:

P1. A polypeptide comprising a modified human fibronectin type III (Fn3) domain, wherein the polypeptide specifically binds to human mesothelin, and wherein BC, DE, and FG loops of the modified Fn3 domain comprise, respectively, the amino acid sequences set forth in:
(a) SEQ ID NOs: 20, 23, and 25;
(b) SEQ ID NOs: 20, 23, and 26;
(c) SEQ ID NOs: 20, 24, and 26;
(d) SEQ ID NOs: 20, 23, and 27;
(e) SEQ ID NOs: 20, 23, and 28;
(f) SEQ ID NOs: 20, 23, and 29;
(g) SEQ ID NOs: 21, 23, and 30;
(h) SEQ ID NOs: 22, 23, and 31; or
(i) SEQ ID NOs: 20, 23, and 32.

P2. The polypeptide of potential claim P1, wherein non-loop regions the Fn3 domain comprise of amino acid sequences having at least 80% identity to corresponding non-loop regions of SEQ ID NO: 1.

P3. The polypeptide of potential claim P1, wherein non-loop regions the Fn3 domain comprise of amino acid sequences having 100% identity to corresponding non-loop regions of SEQ ID NO: 1.

P4. The polypeptide according to any one of the preceding potential claims, wherein the polypeptide binds to mesothelin with a K_{D} of less than 100 nM.

P5. The polypeptide according to any one of the preceding potential claims, wherein the polypeptide binds to mesothelin with a K_{D} of less than 20 nM.

P6. A polypeptide, comprising a human fibronectin type III (Fn3) domain, that binds to mesothelin with a K_{D} of less than 20 nM, wherein the polypeptide comprises SEQ ID NO: 8.

P7. A polypeptide, comprising a human fibronectin type III (Fn3) domain, that binds to mesothelin with a K_{D} of less than 20 nM, wherein the polypeptide comprises SEQ ID NO: 7.

P8. A polypeptide, comprising a human fibronectin type III (Fn3) domain, that binds to mesothelin with a K_{D} of less than 20 nM, wherein the polypeptide consists of SEQ ID NO: 8.

P9. A polypeptide, comprising a human fibronectin type III (Fn3) domain, that binds to mesothelin with a K_{D} of less than 20 nM, wherein the polypeptide consists of SEQ ID NO: 7.

P10. A polypeptide comprising a human fibronectin type III (Fn3) domain, wherein (a) non-loop regions of the Fn3 domain comprise of amino acid sequences having at least 80% identity to corresponding non-loop regions of an Fn3 sequence selected from the group consisting of SEQ ID NOs: 6-19 and (b) BC, DE, and FG loops of the Fn3 domain comprise of amino acid sequences 100% identical to corresponding BC, DE, and FG loops of the Fn3 sequence.

P11. The polypeptide of potential claim P10, wherein the non-loop regions of the Fn3 domain comprise of amino acid sequences having 100% identity to the corresponding non-loop regions of the Fn3 sequence.

P12. A composition comprising the polypeptide according to any one of the preceding potential claims and a pharmaceutically acceptable carrier.

P13. A kit comprising the polypeptide according to any one of potential claims P1-11 and instructions for use.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A polypeptide comprising a modified human fibronectin type III (Fn3) domain, wherein the polypeptide specifically binds to human mesothelin, and wherein BC, DE, and FG loops of the modified Fn3 domain comprise, respectively, the amino acid sequences set forth in:
(a) SEQ ID NOs: 20, 23, and 25;
(b) SEQ ID NOs: 20, 23, and 26;
(c) SEQ ID NOs: 20, 24, and 26;
(d) SEQ ID NOs: 20, 23, and 27;
(e) SEQ ID NOs: 20, 23, and 28;
(f) SEQ ID NOs: 20, 23, and 29;
(g) SEQ ID NOs: 21, 23, and 30;
(h) SEQ ID NOs: 22, 23, and 31; or
(i) SEQ ID NOs: 20, 23, and 32.

2. The polypeptide of claim 1, wherein each non-loop region of the Fn3 domain comprises an amino acid sequence having at least 85% identity to a corresponding non-loop region of SEQ ID NO: 1.

3. The polypeptide of claim 1, wherein non-loop regions the Fn3 domain comprise of amino acid sequences having 100% identity to corresponding non-loop regions of SEQ ID NO: 1.

4. The polypeptide of claim 1, wherein the polypeptide binds to mesothelin with a K_{D} of less than 100 nM.

5. The polypeptide of claim 1, wherein the polypeptide binds to mesothelin with a K_{D} of less than 20 nM.

6. A polypeptide, comprising a human fibronectin type III (Fn3) domain, that binds to mesothelin with a K_{D} of less than 20 nM, wherein the polypeptide comprises SEQ ID NO: 8.

7. A polypeptide, comprising a human fibronectin type III (Fn3) domain, that binds to mesothelin with a K_{D} of less than 20 nM, wherein the polypeptide comprises SEQ ID NO: 7.

8. A polypeptide, comprising a human fibronectin type III (Fn3) domain, that binds to mesothelin with a K_{D} of less than 20 nM, wherein the polypeptide consists of SEQ ID NO: 8.

9. A polypeptide, comprising a human fibronectin type III (Fn3) domain, that binds to mesothelin with a K_{D} of less than 20 nM, wherein the polypeptide consists of SEQ ID NO: 7.

10. A polypeptide comprising a human fibronectin type III (Fn3) domain, wherein (a) each non-loop region of the Fn3 domain comprises an amino acid sequence having at least 85% identity to a corresponding non-loop region of an Fn3 sequence selected from the group consisting of SEQ ID NOs: 7-19 and (b) BC, DE, and FG loops of the Fn3 domain comprise of amino acid sequences 100% identical to corresponding BC, DE, and FG loops of the Fn3 sequence.

11. The polypeptide of claim 10, wherein the non-loop regions of the Fn3 domain comprise of amino acid sequences having 100% identity to the corresponding non-loop regions of the Fn3 sequence.

12. A composition comprising the polypeptide of claim 1 and a pharmaceutically acceptable carrier.

13. A kit comprising the polypeptide of claim 1 and instructions for use.
